SEQUENCE LISTING (69 sequences)

SEQ ID NO: 1 - DNA, 15 bp, Artificial Sequence
  /note=“Description of Artificial Sequence Synthetic oligonucleotide”
acaaccttacttaac

SEQ ID NO: 2 - DNA, 15 bp, Artificial Sequence
  /note=“Description of Artificial Sequence Synthetic oligonucleotide”
acaacctttcttaac

SEQ ID NO: 3 - DNA, 15 bp, Artificial Sequence
  /note=“Description of Artificial Sequence Synthetic oligonucleotide”
acaaccctacttaac

SEQ ID NO: 4 - DNA, 15 bp, Artificial Sequence
  /note=“Description of Artificial Sequence Synthetic oligonucleotide”
acaacctaacttaac

SEQ ID NO: 5 - DNA, 15 bp, Artificial Sequence
  /note=“Description of Artificial Sequence Synthetic oligonucleotide”
acaacgttacataac

SEQ ID NO: 6 - DNA, 32 bp, Artificial Sequence
  /note=“Description of Artificial Sequence Synthetic oligonucleotide”
actcatcacttaccacccattaggactacagc

SEQ ID NO: 7 - DNA, 32 bp, Artificial Sequence; identical to SEQ ID NO: 6
  /note=“Description of Artificial Sequence Synthetic oligonucleotide”

SEQ ID NO: 8 - DNA, 32 bp, Artificial Sequence
  /note=“Description of Artificial Sequence Synthetic oligonucleotide”
actcatcacttaccacccattagcactacagc

SEQ ID NO: 9 - DNA, 32 bp, Artificial Sequence
  /note=“Description of Artificial Sequence Synthetic oligonucleotide”
ttcattaccattcatcccattaggactacagc

SEQ ID NO: 10 - DNA, 32 bp, Artificial Sequence; identical to SEQ ID NO: 9
  /note=“Description of Artificial Sequence Synthetic oligonucleotide”

SEQ ID NO: 11 - DNA, 32 bp, Artificial Sequence; identical to SEQ ID NO: 9
  /note=“Description of Artificial Sequence Synthetic oligonucleotide”

SEQ ID NO: 12 - DNA, 32 bp, Artificial Sequence; identical to SEQ ID NO: 9
  /note=“Description of Artificial Sequence Synthetic oligonucleotide”

SEQ ID NO: 13 - DNA, 32 bp, Artificial Sequence
  /note=“Description of Artificial Sequence Synthetic oligonucleotide”
caaccccttctacaacccattagcactacagc

SEQ ID NO: 14 - DNA, 32 bp, Artificial Sequence; identical to SEQ ID NO: 13
  /note=“Description of Artificial Sequence Synthetic oligonucleotide”

SEQ ID NO: 15 - DNA, 32 bp, Artificial Sequence; identical to SEQ ID NO: 13
  /note=“Description of Artificial Sequence Synthetic oligonucleotide”

SEQ ID NO: 16 - DNA, 17 bp, Artificial Sequence
  /note=“Description of Artificial Sequence Synthetic oligonucleotide”
cccattagcactacagc

SEQ ID NO: 17 - DNA, 17 bp, Artificial Sequence
  /note=“Description of Artificial Sequence Synthetic oligonucleotide”
cccattaggactacagc

SEQ ID NO: 18 - DNA, 52 bp, Artificial Sequence
  /note=“Description of Artificial Sequence Synthetic primer”
ggatattcctttctactctttgacatcatctatcacgtggctctcaccaccc

SEQ ID NO: 19 - DNA, 57 bp, Artificial Sequence
  /note=“Description of Artificial Sequence Synthetic primer”
ggatattcctttctactctttgacatcatctatcaaacaggacgaactggatttcct

SEQ ID NO: 20 - DNA, 53 bp, Artificial Sequence
  /note=“Description of Artificial Sequence Synthetic primer”
ggatattcctttctactctttgacatcatctatcatcaccccaatgcagcgaa

SEQ ID NO: 21 - DNA, 57 bp, Artificial Sequence
  /note=“Description of Artificial Sequence Synthetic primer”
ggatattcctttctactctttgacatcatctatcagagcacagtcactttgactcac

SEQ ID NO: 22 - DNA, 61 bp, Artificial Sequence
  /note=“Description of Artificial Sequence Synthetic primer”
ggatattcctttctactctttgacatcatctatcacagtctttactcacctgtagatgtct

SEQ ID NO: 23 - DNA, 63 bp, Artificial Sequence
  /note=“Description of Artificial Sequence Synthetic primer”
ggatattcctttctactctttgacatcatctatcattcatgaagacctcacagtaaaaatagg

SEQ ID NO: 24 - DNA, 59 bp, Artificial Sequence
  /note=“Description of Artificial Sequence Synthetic primer”
ggatattcctttctactctttgacatcatctatcaggagattcctgatgggcagattac

SEQ ID NO: 25 - DNA, 68 bp, Artificial Sequence
  /note=“Description of Artificial Sequence Synthetic primer”
ggatattcctttctactctttgacatcatctatcagttattctgatttcccattcttttctttactta

SEQ ID NO: 26 - DNA, 57 bp, Artificial Sequence
  /note=“Description of Artificial Sequence Synthetic primer”
ggatattcctttctactctttgacatcatctatcattaccttatacaccgtgccgaa

SEQ ID NO: 27 - DNA, 62 bp, Artificial Sequence
  /note=“Description of Artificial Sequence Synthetic primer”
ggatattcctttctactctttgacatcatctatcatcccagaaggtgagaaagttaaaattc

SEQ ID NO: 28 - DNA, 54 bp, Artificial Sequence
  /note=“Description of Artificial Sequence Synthetic primer”
cctatggtagttaaatgtacattggatattcctttctactctttgacatcatct

SEQ ID NO: 29 - DNA, 60 bp, Artificial Sequence
  /note=“Description of Artificial Sequence Synthetic primer”
agacgtgtgctcttccgatctcaathhhhhhhhhhhhhhhccgctccttgtagccaatga

SEQ ID NO: 30 - DNA, 60 bp, Artificial Sequence
  /note=“Description of Artificial Sequence Synthetic primer”
agacgtgtgctcttccgatctatcahhhhhhhhhhhhhhhctctccaggttctttggggg

SEQ ID NO: 31 - DNA, 61 bp, Artificial Sequence
  /note=“Description of Artificial Sequence Synthetic primer”
agacgtgtgctcttccgatctcaathhhhhhhhhhhhhhhagatttgcccagactcagctc

SEQ ID NO: 32 - DNA, 58 bp, Artificial Sequence
  /note=“Description of Artificial Sequence Synthetic primer”
agacgtgtgctcttccgatctatcahhhhhhhhhhhhhhhgctcgggacattgcctgt

SEQ ID NO: 33 - DNA, 59 bp, Artificial Sequence
  /note=“Description of Artificial Sequence Synthetic primer”
agacgtgtgctcttccgatctcaathhhhhhhhhhhhhhhgctgccagaaactgcctct

SEQ ID NO: 34 - DNA, 64 bp, Artificial Sequence
  /note=“Description of Artificial Sequence Synthetic primer”
agacgtgtgctcttccgatctcaathhhhhhhhhhhhhhhccacaaaatggatccagacaactg

SEQ ID NO: 35 - DNA, 67 bp, Artificial Sequence
  /note=“Description of Artificial Sequence Synthetic primer”
agacgtgtgctcttccgatctatcahhhhhhhhhhhhhhhacaatgtcaccacattacatacttacc

SEQ ID NO: 36 - DNA, 63 bp, Artificial Sequence
  /note=“Description of Artificial Sequence Synthetic primer”
agacgtgtgctcttccgatctatcahhhhhhhhhhhhhhhgacaaaaggtgaaagtctcccac

SEQ ID NO: 37 - DNA, 59 bp, Artificial Sequence
  /note=“Description of Artificial Sequence Synthetic primer”
agacgtgtgctcttccgatctatcahhhhhhhhhhhhhhhgctcccaaccaagctctct

SEQ ID NO: 38 - DNA, 63 bp, Artificial Sequence
  /note=“Description of Artificial Sequence Synthetic primer”
agacgtgtgctcttccgatctcaathhhhhhhhhhhhhhhgcaaagcagaaactcacatcgag

SEQ ID NO: 39 - DNA, 32 bp, Artificial Sequence
  /note=“Description of Artificial Sequence Synthetic primer”
gactggagttcagacgtgtgctcttccgatct

SEQ ID NO: 40 - DNA, 16 bp, Artificial Sequence
  /note=“Description of Artificial Sequence Synthetic primer”
ccacccgcacgtctgt

SEQ ID NO: 41 - DNA, 21 bp, Artificial Sequence
  /note=“Description of Artificial Sequence Synthetic primer”
ggatttcctcatggaagccct

SEQ ID NO: 42 - DNA, 22 bp, Artificial Sequence
  /note=“Description of Artificial Sequence Synthetic primer”
gcgaacaatgttctggtggttg

SEQ ID NO: 43 - DNA, 20 bp, Artificial Sequence
  /note=“Description of Artificial Sequence Synthetic primer”
actttgactcaccggtggat

SEQ ID NO: 44 - DNA, 18 bp, Artificial Sequence
  /note=“Description of Artificial Sequence Synthetic primer”
cgggccatcccgaagtct

SEQ ID NO: 45 - DNA, 28 bp, Artificial Sequence
  /note=“Description of Artificial Sequence Synthetic primer”
cctcacagtaaaaataggtgattttggt

SEQ ID NO: 46 - DNA, 26 bp, Artificial Sequence
  /note=“Description of Artificial Sequence Synthetic primer”
gattacagtgggacaaagaattggat

SEQ ID NO: 47 - DNA, 21 bp, Artificial Sequence
  /note=“Description of Artificial Sequence Synthetic primer”
agggcaagttcactacagcaa

SEQ ID NO: 48 - DNA, 16 bp, Artificial Sequence
  /note=“Description of Artificial Sequence Synthetic primer”
gccgaacgcaccggag

SEQ ID NO: 49 - DNA, 25 bp, Artificial Sequence
  /note=“Description of Artificial Sequence Synthetic primer”
agaaagttaaaattcccgtcgctat

SEQ ID NO: 50 - DNA, 29 bp, Artificial Sequence
  /note=“Description of Artificial Sequence Synthetic primer”
acgtctgtaggggagtacatcaagaccga

SEQ ID NO: 51 - DNA, 28 bp, Artificial Sequence
  /note=“Description of Artificial Sequence Synthetic primer”
aagccctgatcatcaggtaaagccacaa

SEQ ID NO: 52 - DNA, 29 bp, Artificial Sequence
  /note=“Description of Artificial Sequence Synthetic primer”
tggtggttgaatttgctgcagagcagaga

SEQ ID NO: 53 - DNA, 27 bp, Artificial Sequence
  /note=“Description of Artificial Sequence Synthetic primer”
ggtggatgaagtggttttcctccaaaa

SEQ ID NO: 54 - DNA, 28 bp, Artificial Sequence
  /note=“Description of Artificial Sequence Synthetic primer”
ccgaagtctccaatcttggccactctct

SEQ ID NO: 55 - DNA, 34 bp, Artificial Sequence
  /note=“Description of Artificial Sequence Synthetic primer”
gtgattttggtctagctacagtgaaatctcgagt

SEQ ID NO: 56 - DNA, 34 bp, Artificial Sequence
  /note=“Description of Artificial Sequence Synthetic primer”
aaagaattggatctggatcatttggaacagtccc

SEQ ID NO: 57 - DNA, 27 bp, Artificial Sequence
  /note=“Description of Artificial Sequence Synthetic primer”
actacagcaagtgatgtgtgggcctga

SEQ ID NO: 58 - DNA, 25 bp, Artificial Sequence
  /note=“Description of Artificial Sequence Synthetic primer”
caccggagcccagcactttgatcta

SEQ ID NO: 59 - DNA, 32 bp, Artificial Sequence
  /note=“Description of Artificial Sequence Synthetic primer”
gtcgctatcaaggaattaagagaagcaacata

SEQ ID NO: 60 - DNA, 36 bp, Artificial Sequence
  /note=“Description of Artificial Sequence Synthetic primer”
acacgacgctcttccgatctccacccgcacgtctgt

SEQ ID NO: 61 - DNA, 41 bp, Artificial Sequence
  /note=“Description of Artificial Sequence Synthetic primer”
acacgacgctcttccgatctggatttcctcatggaagccct

SEQ ID NO: 62 - DNA, 42 bp, Artificial Sequence
  /note=“Description of Artificial Sequence Synthetic primer”
acacgacgctcttccgatctgcgaacaatgttctggtggttg

SEQ ID NO: 63 - DNA, 40 bp, Artificial Sequence
  /note=“Description of Artificial Sequence Synthetic primer”
acacgacgctcttccgatctactttgactcaccggtggat

SEQ ID NO: 64 - DNA, 38 bp, Artificial Sequence
  /note=“Description of Artificial Sequence Synthetic primer”
acacgacgctcttccgatctcgggccatcccgaagtct

SEQ ID NO: 65 - DNA, 48 bp, Artificial Sequence
  /note=“Description of Artificial Sequence Synthetic primer”
acacgacgctcttccgatctcctcacagtaaaaataggtgattttggt

SEQ ID NO: 66 - DNA, 46 bp, Artificial Sequence
  /note=“Description of Artificial Sequence Synthetic primer”
acacgacgctcttccgatctgattacagtgggacaaagaattggat

SEQ ID NO: 67 - DNA, 41 bp, Artificial Sequence
  /note=“Description of Artificial Sequence Synthetic primer”
acacgacgctcttccgatctagggcaagttcactacagcaa

SEQ ID NO: 68 - DNA, 36 bp, Artificial Sequence
  /note=“Description of Artificial Sequence Synthetic primer”
acacgacgctcttccgatctgccgaacgcaccggag

SEQ ID NO: 69 - DNA, 45 bp, Artificial Sequence
  /note=“Description of Artificial Sequence Synthetic primer”
acacgacgctcttccgatctagaaagttaaaattcccgtcgctat

(19) United States
(12) Patent Application Publication
(10) Pub. No.: US 2023/0399687 A1
(43) Pub. Date: Dec. 14, 2023

(54) Quantitative Multiplex Amplicon Sequencing System

(71) Applicants: NUPROBE USA, INC., Houston, TX (US); WILLIAM MARSH RICE UNIVERSITY, Houston, TX (US)

(72) Inventors: David Y. ZHANG, Houston, TX (US); Peng DAI, Houston, TX (US); Pengying HAO, Houston, TX (US); Alessandro PINTO, Houston, TX (US)

(73) Assignees: NUPROBE USA, INC., Houston, TX (US); WILLIAM MARSH RICE UNIVERSITY, Houston, TX (US)

(21) Appl. No.: 18/034,753

(22) PCT Filed: Nov. 1, 2021
(86) PCT No.: PCT/US2021/057573

§ 371 Date: May 1, 2023

Related U.S. Application Data
(60) Provisional application No. 63/108,649, filed on Nov. 2, 2020.

(51) Int. Cl. C12Q 1/6858

(52) U.S. Cl. CPC C12Q 1/6858

(57) ABSTRACT

The present invention discloses methods of quantitative multiplex amplicon sequencing system for labeling the original DNA sample with an oligonucleotide barcode sequence by polymerase chain reaction, amplifying the genomic region(s) for high-throughput sequencing and quantifying the sequence in DNA sample. The methods allow analyzing a DNA sample comprising between 1 and 10,000 Target Regions for quantifying potential sequence variants and wildtype molecules.

### CROSS-REFERENCE TO RELATED APPLICATIONS

This application claims the benefit of U.S. Provisional Patent Application No. 63/108,649, filed Nov. 2, 2020, which is incorporated by reference herein in its entirety.

### FIELD

The present disclosure relates to the fields of molecular biology and bioinformatics. More particularly, it relates to methods for analyzing DNA samples to quantify potential sequence variants and wildtype molecules.

### INCORPORATION OF SEQUENCE LISTING

The instant application contains a Sequence Listing which has been submitted electronically in ASCII format and is hereby incorporated by reference in its entirety. Said ASCII copy, created on Oct. 7, 2021, is named P35008W000SL.txt and is 24,576 bytes in size measured in Microsoft Windows®.

### BACKGROUND

Detecting DNA variants with low allele frequency is difficult due to the presence of polymerase error during polymerase chain reaction (PCR) amplification and sequencing error. Although low frequency mutations, such as cancer mutations and pathogen drug resistance mutations, hold important clinical and biological information, standard next generation sequencing (NGS) cannot confidently identify variants with variant allele frequencies (VAF) below approximately 2% to 5%. 100051 Here, methods for attaching unique molecular identifiers (UMI) to original nucleic acid molecules to accurately identify rare mutations with a logarithm of odds (LOD) down to 0.1% are provided. A method based on Blocker Displacement Amplification (BDA) that enriches variant sequences over wildtype molecules to achieve accurate quantitation with low-depth sequencing is also provided.

### SUMMARY

In one aspect, this disclosure provides a method for analyzing a DNA sample comprising at least one Target Region for potential sequence variants, the method comprising: (a) contacting the DNA sample with: (i) a set of unique molecular identifier (UMI) Primers, where each UMI primer comprises a UMI sequence and a gene-specific sequence that is complementary to a Target Region subsequence; (ii) a first DNA polymerase; and (iii) reagents and buffers needed for DNA polymerase extension to generate a mixture; (b) subjecting the mixture of step (a) to one or more temperatures that allow primer binding and DNA polymerase extension; (c) removing non-extended UMI primers to produce a product; (d) mixing the product of step (c) with: (i) a second set of DNA primers; (ii) a second DNA polymerase; and (iii) reagents and buffers needed for a polymerase chain reaction (PCR), and performing PCR to produce a PCR product; (e) subjecting the PCR product produced in step (d) to high-throughput DNA sequencing and obtaining a sequence file comprising next generation sequencing (NGS) reads; (0 identifying a vetoed UMI sequence, where at least 1, 2, 3, 4, 5, 6, 7, 8, 9, or 10 NGS reads containing the vetoed UMI sequence also comprise a wildtype sequence of the at least one Target Region; (g) removing from consideration all NGS reads comprising the vetoed UMI sequence identified in step (f); and (h) generating a sequence variant call by quantifying DNA variant molecules based on bioinformatic analysis of the NGS reads that are not removed in step (g).

In one aspect, this disclosure provides a method for analyzing a DNA sample comprising at least one Target Region for potential sequence variants, the method comprising: (a) preparing a next generation sequencing (NGS) library, where a unique molecular identifier (UMI) sequence is added to a plurality of polynucleotides present in the NGS library; (b) obtaining a sequence file comprising NGS reads; (c) identifying a vetoed UMI sequence, where at least 1, 2, 3, 4, 5, 6, 7, 8, 9, or 10 NGS reads containing the vetoed UMI sequence also comprise a wildtype sequence of the at least one Target Region; (d) removing from consideration all NGS reads comprising the vetoed UMI sequence identified in step (c); and (e) generating a sequence variant call by quantifying DNA variant molecules based on bioinformatic analysis of the NGS reads that are not removed in step (d).

In one aspect, this disclosure provides a method comprising: (a) amplifying a population of distinct initial target DNA molecules from a tagged genomic sample thereby producing a population of amplified target DNA molecules, where the distinct initial target DNA molecules that comprise a polymorphic target sequence are tagged with different unique molecular identifier (UMI) sequences, where the UMI sequences comprise at least one nucleotide base selected from: R, Y, S, W, K, M, B, D, H, V, N and modified versions thereof, and where each of a plurality of the amplified target DNA molecules comprises the polymorphic target sequence and an associated UMI sequence of the different UMI sequences; (b) sequencing the plurality of the amplified target DNA molecules, thereby producing a plurality of NGS sequence reads, where the sequencing step provides, for each of the amplified target DNA molecules that are sequenced: the nucleotide sequence of: (i) at least a portion of the polymorphic target sequence; and (ii) an associated UMI sequence of the UMI sequences; (c) identifying a vetoed UMI sequence, where at least 1, 2, 3, 4, 5, 6, 7, 8, 9, or 10 NGS reads containing the vetoed UMI sequence also comprise a WT sequence of the at least one Target Region; (d) removing from consideration all NGS reads comprising the vetoed UMI sequence identified in step (c); and generating a sequence variant call by quantifying DNA variant molecules based on bioinformatic analysis of the NGS reads that are not removed in step (d).

In one aspect, this disclosure provides a method to analyze nucleic acid sequences, the method comprising: (a) attaching a unique molecular identifier (UMI) from a pool of UMIs to a first end of each strand of a plurality of analyte nucleic acid fragments to form a plurality of uniquely identified analyte nucleic acid fragments where the pool of UMIs is in excess of the plurality of analyte nucleic acid fragments; (b) redundantly determining nucleotide sequence of a uniquely identified analyte nucleic acid fragment to generate next generation sequencing (NGS) reads, where determined nucleotide sequences which share a UMI form a UMI Family; (c) identifying a vetoed UMI sequence, where at least 1, 2, 3, 4, 5, 6, 7, 8, 9, or 10 NGS reads containing the vetoed UMI sequence also comprise a WT sequence of the at least one Target Region; (d) removing from consideration all NGS reads comprising the vetoed UMI sequence identified in step (c); and (e) generating a sequence variant call by quantifying DNA variant molecules based on bioinformatic analysis of the NGS reads that are not removed in step (d).

In one aspect, this disclosure provides a method for analyzing a DNA sample comprising at least one Target Region for potential sequence variants, the method comprising: (a) contacting the DNA sample with: (i) a set of unique molecular identifier (UMI) Primers, where each UMI primer comprises a UMI sequence and a gene-specific sequence that is complementary to a Target Region subsequence; (ii) a first DNA polymerase; and (iii) reagents and buffers needed for DNA polymerase extension to generate a mixture; (b) subjecting the mixture of step (a) to one or more temperatures that allow primer binding and DNA polymerase extension; (c) removing non-extended UMI primers to produce a product; (d) mixing the product of step (c) with: (i) a second set of DNA primers; (ii) a second DNA polymerase; and (iii) reagents and buffers needed for a polymerase chain reaction (PCR), and performing PCR to produce a PCR product; (e) subjecting the PCR product produced in step (d) to high-throughput DNA sequencing and obtaining a sequence file comprising next generation sequencing (NGS) reads; (f) grouping the NGS reads into at least one UMI Family, where each NGS read within a UMI Family comprises an identical UMI sequence and aligns to the same amplicon; (g) removing from consideration, for each amplicon, all GNS reads in a below-threshold UMI Family, where the below-threshold UMIT Family comprises a size smaller than X, where X is Y % of the mean value for the largest Z UMI Family sizes for the amplicon, where Y is between 1% and 20%, and where Z is between 1 and 20; and (h) generating a sequence variant call based on bioinformatic analysis of the NGS reads that were not removed in step (g).

In one aspect, this disclosure provides a method for analyzing a DNA sample comprising at least one Target Region for potential sequence variants, the method comprising: (a) preparing a next generation sequencing (NGS) library, where a unique molecular identifier (UMI) sequence is added to a plurality of polynucleotides present in the NGS library; (b) obtaining a sequence file comprising NGS reads; (c) grouping the NGS reads into at least one UMI Family, where each NGS read within a UMI Family comprises an identical UMI sequence and aligns to the same amplicon; (d) removing from consideration, for each amplicon, all GNS reads in a below-threshold UMI Family, where the below-threshold UMIT Family comprises a size smaller than X, where X is Y % of the mean value for the largest Z UMI Family sizes for the amplicon, where Y is between 1% and 20%, and where Z is between 1 and 20; and (e) generating a sequence variant call based on bioinformatic analysis of the NGS reads that were not removed in step (d).

In one aspect, this disclosure provides a method of sequencing comprising: (a) amplifying a population of distinct initial target DNA molecules from a tagged genomic sample thereby producing a population of amplified target DNA molecules, where the distinct initial target DNA molecules that comprise a polymorphic target sequence are tagged with different unique molecular identifier (UMI) sequences, where the UMI sequences comprise at least one nucleotide base selected from: R, Y, S, W, K, M, B, D, H, V, N and modified versions thereof, and where each of a plurality of the amplified target DNA molecules comprises the polymorphic target sequence and an associated UMI sequence of the different UMI sequences; (b) sequencing the plurality of the amplified target DNA molecules, thereby producing a plurality of NGS sequence reads, where the sequencing step provides, for each of the amplified target DNA molecules that are sequenced: the nucleotide sequence of: (i) at least a portion of the polymorphic target sequence; and (ii) an associated UMI sequence of the UMI sequences; (c) grouping the NGS reads into at least one UMI Family, where each NGS read within a UMI Family comprises an identical UMI sequence and aligns to the same polymorphic target sequence; (d) removing from consideration, for each polymorphic target sequence, all NGS reads in a below-threshold UMI Family; where the below-threshold UMI Family comprises a size smaller than X, where X is Y % of the mean value for the largest Z UMI Family sizes for the amplicon, where Y is between 1% and 20%, and where Z is between 1 and 20; and (e) generating a sequence variant call based on bioinformatic analysis of the NGS reads that were not removed in step (d).

In one aspect, this disclosure provides a method to analyze nucleic acid sequences, the method comprising: (a) attaching a unique molecular identifier (UMI) from a pool of UMIs to a first end of each strand of a plurality of analyte nucleic acid fragments to form a plurality of uniquely identified analyte nucleic acid fragments where the pool of UMIs is in excess of the plurality of analyte nucleic acid fragments; (b) redundantly determining nucleotide sequence of a uniquely identified analyte nucleic acid fragment, to generate next generation sequencing (NGS) reads where determined nucleotide sequences which share a UMI form a UMI Family; (c) removing from consideration, for each polymorphic target sequence, all NGS reads in a below-threshold UMI Family; where the below-threshold UMI Family comprises a size smaller than X, where X is Y % of the mean value for the largest Z UMI Family sizes for the amplicon, where Y is between 1% and 20%, and where Z is between 1 and 20; and (d) generating a sequence variant call based on bioinformatic analysis of the NGS reads that were not removed in step (c).

In one aspect, this disclosure provides a method for analyzing a DNA sample comprising at least one Target Region for potential sequence variants, the method comprising: (a) contacting the DNA sample with: (i) a set of unique molecular identifier (UMI) Primers, where each UMI primer comprises a UMI sequence and a gene-specific sequence that is complementary to a Target Region subsequence; (ii) a first DNA polymerase; and (iii) reagents and buffers needed for DNA polymerase extension to generate a mixture; (b) subjecting the mixture of step (a) to one or more temperatures that allow primer binding and DNA polymerase extension; (c) removing non-extended UMI primers to produce a product; (d) mixing the product of step (c) with: (i) a second set of DNA primers; (ii) a second DNA polymerase; and (iii) reagents and buffers needed for a polymerase chain reaction (PCR), and performing PCR to produce a PCR product; (e) subjecting the PCR product produced in step (d) to high-throughput DNA sequencing and obtaining a sequence file comprising next generation sequencing (NGS) reads; (f) grouping the NGS reads into at least a first UMI Family and a second UMI Family, where each NGS read within the first UMI Family comprises an identical UMI sequence and aligns to a common amplicon, where each NGS read within the second UMI Family comprises an identical UMI sequence and aligns to the common amplicon, and where the UMI sequence of the first UMI Family differs by 1 nucleotide or 2 nucleotides as compared to the UMI sequence of the second UMI Family; (g) removing from consideration the NGS reads in the UMI Family that has the fewest NGS reads between the first UMI Family and the second UMI Family; and (h) generating a sequence variant call based on bioinformatic analysis of the NGS reads that were not removed in step (g).

In one aspect, this disclosure provides a method for analyzing a DNA sample comprising at least one Target Region for potential sequence variants, the method comprising: (a) preparing a next generation sequencing (NGS) library, where a unique molecular identifier (UMI) sequence is added to a plurality of polynucleotides present in the NGS library; (b) obtaining a sequence file comprising NGS reads; (c) grouping the NGS reads into at least a first UMI Family and a second UMI Family, where each NGS read within the first UMI Family comprises an identical UMI sequence and aligns to a common amplicon, where each NGS read within the second UMI Family comprises an identical UMI sequence and aligns to the common amplicon, and where the UMI sequence of the first UMI Family differs by 1 nucleotide or 2 nucleotides as compared to the UMI sequence of the second UMI Family; (d) removing from consideration the NGS reads in the UMI Family that has the fewest NGS reads between the first UMI Family and the second UMI Family; and (e) generating a sequence variant call based on bioinformatic analysis of the NGS reads that were not removed in step (d).

In one aspect, this disclosure provides a method of sequencing, the method comprising: (a) amplifying a population of distinct initial target DNA molecules from a tagged genomic sample thereby producing a population of amplified target DNA molecules, where the distinct initial target DNA molecules that comprise a polymorphic target sequence are tagged with different unique molecular identifier (UMI) sequences, where the UMI sequences comprise at least one nucleotide base selected from: R, Y, S, W, K, M, B, D, H, V, N and modified versions thereof, and where each of a plurality of the amplified target DNA molecules comprises the polymorphic target sequence and an associated UMI sequence of the different UMI sequences; (b) sequencing the plurality of the amplified target DNA molecules, thereby producing a plurality of NGS sequence reads, where the sequencing step provides, for each of the amplified target DNA molecules that are sequenced: the nucleotide sequence of: (i) at least a portion of the polymorphic target sequence; and (ii) an associated UMI sequence of the UMI sequences; (c) grouping the NGS reads into at least a first UMI Family and a second UMI Family, where each NGS read within the first UMI Family comprises an identical UMI sequence and aligns to the polymorphic target sequence, where each NGS read within the second UMI Family comprises an identical UMI sequence and aligns to the polymorphic target sequence, and where the UMI sequence of the first UMI Family differs by 1 nucleotide or 2 nucleotides as compared to the UMI sequence of the second UMI Family; (d) removing from consideration the NGS reads in the UMI Family that has the fewest NGS reads between the first UMI Family and the second UMI Family; and (e) generating a sequence variant call based on bioinformatic analysis of the NGS reads that were not removed in step (d).

In one aspect, this disclosure provides a method to analyze nucleic acid sequences, the method comprising: (a) attaching a unique molecular identifier (UMI) from a pool of UMIs to a first end of each strand of a plurality of analyte nucleic acid fragments to form a plurality of uniquely identified analyte nucleic acid fragments where the pool of UMIs is in excess of the plurality of analyte nucleic acid fragments; (b) redundantly determining nucleotide sequence of a uniquely identified analyte nucleic acid fragment to generate next generation sequencing (NGS) reads, where determined nucleotide sequences which share a UMI form a UMI Family; (c) grouping the determined nucleotide sequences into at least a first UMI Family and a second UMI Family, where each determined nucleotide sequence within the first UMI Family comprises an identical UMI sequence and aligns to a common amplicon, where each determined nucleotide sequence within the second UMI Family comprises an identical UMI sequence and aligns to the common amplicon, and where the UMI sequence of the first UMI Family differs by 1 nucleotide or 2 nucleotides as compared to the UMI sequence of the second UMI Family; (d) removing from consideration the NGS reads in the UMI Family that has the fewest determined nucleotide sequences between the first UMI Family and the second UMI Family; and (e) generating a sequence variant call based on bioinformatic analysis of the remaining determined nucleotide sequences.

### BRIEF DESCRIPTION OF THE DRAWINGS

FIG. 1 depicts a schematic of next generation sequencing (NGS) library preparation. UMI: Unique molecular identifier; NGS: Next generation sequencing.

FIG. 2 depicts a non-limiting embodiment of the application that is discussed in Example 1.

FIG. 3 depicts a schematic of a quantitative blocker displacement amplification (QBDA) workflow that enriches variant sequences over wildtype sequences.

FIG. 4 depicts a quantitative amplicon sequencing (QASeq) workflow, where there is no sequence preference during amplification.

FIG. 5 depicts a schematic of a QBDA analysis workflow. The three modules (e.g., WTveto; Nearest Neighbor Check; Dynamic Cutoff) can be performed in any order or in any combination for data analysis.

FIG. 6 depicts a schematic of Nearest Neighbor Check with a Distance Threshold of 1.

FIG. 7 depicts a schematic of WTveto.

FIG. 8 comprises panels A, B, and C. FIG. 8 depicts an illustration of Dynamic Cutoff for two mutations with different unique molecular identifier (UMI) family size distributions. Panel A depicts overall UMI family size distribution for mutation 1 (black) and mutation 2 (gray). The area highlighted in gray in panel A is expanded for mutation 1 in panel B and for mutation 2 in panel C.

FIG. 9 depicts the assignment of top genotypes to unique molecular identifiers (UMI) for a non-small cell lung cancer (NSCLC) QBDA panel.

FIG. 10 comprises panels A and B. FIG. 10 depicts that unique molecular identifier (UMI) quantitation by Dynamic Cutoff (panel A) is sequencing read depth independent, in contrast to UMI quantitation without any cutoff measures (panel B). Analysis of the NSCLC QBDA panel sequencing data was performed for the full dataset of 1 million (1M) reads and on a sub-sample generated by random down-sampling to 600,000 (600K) reads.

FIG. 11 depicts unique molecular identifier (UMI) quantitation of 30 ng of NSCLC panel gBlock spike-in standards with UMI correction (Dynamic Cutoff and Nearest Neighbor Check) versus no UMI correction.

FIG. 12 depicts an alternative QBDA workflow. As compared to FIG. 3, the alternative QBDA workflow eliminates the universal PCR amplification step and eliminates purification after BDA amplification.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entirety.

Any composition provided herein is specifically envisioned for use with any applicable method provided herein.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to inclusive of the edges of the range as well as any number between the defined edges of the range. For example, “between 1 and 10” includes any number between 1 and 10, as well as the number 1 and the number 10.

As used herein, the singular form “a,” “an,” and “the” include plural references unless the context clearly dictates otherwise. For example, the term “a compound” or “at least one compound” may include a plurality of compounds, including mixtures thereof. As used herein, the term “plurality” refers to any number greater than one.

This disclosure provides methods for detecting rare DNA variants from a variety of sample sizes. This disclosure provides three distinct workflows that can be used alone, or in any combination to detect and/or quantify DNA variants: WTveto, Nearest Neighbor Check, and Dynamic Cutoff. For each method, sequencing data comprising sequence reads that each contain a unique molecular identifier (UMI) are obtained. For WTveto, a particular UMI may be assigned to a wildtype (WT) genotype when more than X copies of WT reads are identified. For Nearest Neighbor Check, UMIs are compared to other UMIs that have related sequences to generate UMI families, and only the largest UMI families are retained. For Dynamic Cutoff, X % of the average top Z UMI family size is determined, and UMIs comprising a family size equal to, or below, the cutoff are discarded.

In an aspect, this disclosure provides a method for analyzing a DNA sample comprising at least one Target Region for potential sequence variants, the method comprising: (a) contacting the DNA sample with: (i) a set of unique molecular identifier (UMI) Primers, where each UMI primer comprises a UMI sequence and a gene-specific sequence that is complementary to a Target Region subsequence; (ii) a first DNA polymerase; and (iii) reagents and buffers needed for DNA polymerase extension to generate a mixture; (b) subjecting the mixture of step (a) to one or more temperatures that allow primer binding and DNA polymerase extension; (c) removing non-extended UMI primers to produce a product; (d) mixing the product of step (c) with: (i) a second set of DNA primers; (ii) a second DNA polymerase; and (iii) reagents and buffers needed for a polymerase chain reaction (PCR), and performing PCR to produce a PCR product; (e) subjecting the PCR product produced in step (d) to high-throughput DNA sequencing and obtaining a sequence file comprising next generation sequencing (NGS) reads; (f) identifying a vetoed UMI sequence, where at least 1, 2, 3, 4, 5, 6, 7, 8, 9, or 10 NGS reads containing the vetoed UMI sequence also comprise a wildtype sequence of the at least one Target Region; (g) removing from consideration all NGS reads comprising the vetoed UMI sequence identified in step (f); and (h) generating a sequence variant call by quantifying DNA variant molecules based on bioinformatic analysis of the NGS reads that are not removed in step (g).

In an aspect, this disclosure provides a method for analyzing a DNA sample comprising at least one Target Region for potential sequence variants, the method comprising: (a) preparing a next generation sequencing (NGS) library, where a unique molecular identifier (UMI) sequence is added to a plurality of polynucleotides present in the NGS library; (b) obtaining a sequence file comprising NGS reads; (c) identifying a vetoed UMI sequence, where at least 1, 2, 3, 4, 5, 6, 7, 8, 9, or 10 NGS reads containing the vetoed UMI sequence also comprise a wildtype sequence of the at least one Target Region; (d) removing from consideration all NGS reads comprising the vetoed UMI sequence identified in step (c); and (e) generating a sequence variant call by quantifying DNA variant molecules based on bioinformatic analysis of the NGS reads that are not removed in step (d).

In an aspect, this disclosure provides a method comprising: (a) amplifying a population of distinct initial target DNA molecules from a tagged genomic sample thereby producing a population of amplified target DNA molecules, where the distinct initial target DNA molecules that comprise a polymorphic target sequence are tagged with different unique molecular identifier (UMI) sequences, where the UMI sequences comprise at least one nucleotide base selected from: R, Y, S, W, K, M, B, D, H, V, N and modified versions thereof, and where each of a plurality of the amplified target DNA molecules comprises the polymorphic target sequence and an associated UMI sequence of the different UMI sequences; (b) sequencing the plurality of the amplified target DNA molecules, thereby producing a plurality of NGS sequence reads, where the sequencing step provides, for each of the amplified target DNA molecules that are sequenced: the nucleotide sequence of: (i) at least a portion of the polymorphic target sequence; and (ii) an associated UMI sequence of the UMI sequences; (c) identifying a vetoed UMI sequence, where at least 1, 2, 3, 4, 5, 6, 7, 8, 9, or 10 NGS reads containing the vetoed UMI sequence also comprise a WT sequence of the at least one Target Region; (d) removing from consideration all NGS reads comprising the vetoed UMI sequence identified in step (c); and generating a sequence variant call by quantifying DNA variant molecules based on bioinformatic analysis of the NGS reads that are not removed in step (d).

In an aspect, this disclosure provides a method to analyze nucleic acid sequences, the method comprising: (a) attaching a unique molecular identifier (UMI) from a pool of UMIs to a first end of each strand of a plurality of analyte nucleic acid fragments to form a plurality of uniquely identified analyte nucleic acid fragments where the pool of UMIs is in excess of the plurality of analyte nucleic acid fragments; (b) redundantly determining nucleotide sequence of a uniquely identified analyte nucleic acid fragment to generate next generation sequencing (NGS) reads, where determined nucleotide sequences which share a UMI form a UMI Family; (c) identifying a vetoed UMI sequence, where at least 1, 2, 3, 4, 5, 6, 7, 8, 9, or 10 NGS reads containing the vetoed UMI sequence also comprise a WT sequence of the at least one Target Region; (d) removing from consideration all NGS reads comprising the vetoed UMI sequence identified in step (c); and (e) generating a sequence variant call by quantifying DNA variant molecules based on bioinformatic analysis of the NGS reads that are not removed in step (d).

In an aspect, this disclosure provides a method for analyzing a DNA sample comprising at least one Target Region for potential sequence variants, the method comprising: (a) contacting the DNA sample with: (i) a set of unique molecular identifier (UMI) Primers, where each UMI primer comprises a UMI sequence and a gene-specific sequence that is complementary to a Target Region subsequence; (ii) a first DNA polymerase; and (iii) reagents and buffers needed for DNA polymerase extension to generate a mixture; (b) subjecting the mixture of step (a) to one or more temperatures that allow primer binding and DNA polymerase extension; (c) removing non-extended UMI primers to produce a product; (d) mixing the product of step (c) with: (i) a second set of DNA primers; (ii) a second DNA polymerase; and (iii) reagents and buffers needed for a polymerase chain reaction (PCR), and performing PCR to produce a PCR product; (e) subjecting the PCR product produced in step (d) to high-throughput DNA sequencing and obtaining a sequence file comprising next generation sequencing (NGS) reads; (f) grouping the NGS reads into at least one UMI Family, where each NGS read within a UMI Family comprises an identical UMI sequence and aligns to the same amplicon; (g) removing from consideration, for each amplicon, all GNS reads in a below-threshold UMI Family, where the below-threshold UMIT Family comprises a size smaller than X, where X is Y % of the mean value for the largest Z UMI Family sizes for the amplicon, where Y is between 1% and 20%, and where Z is between 1 and 20; and (h) generating a sequence variant call based on bioinformatic analysis of the NGS reads that were not removed in step (g).

In an aspect, this disclosure provides a method for analyzing a DNA sample comprising at least one Target Region for potential sequence variants, the method comprising: (a) preparing a next generation sequencing (NGS) library, where a unique molecular identifier (UMI) sequence is added to a plurality of polynucleotides present in the NGS library; (b) obtaining a sequence file comprising NGS reads; (c) grouping the NGS reads into at least one UMI Family, where each NGS read within a UMI Family comprises an identical UMI sequence and aligns to the same amplicon; (d) removing from consideration, for each amplicon, all GNS reads in a below-threshold UMI Family, where the below-threshold UMIT Family comprises a size smaller than X, where X is Y % of the mean value for the largest Z UMI Family sizes for the amplicon, where Y is between 1% and 20%, and where Z is between 1 and 20; and (e) generating a sequence variant call based on bioinformatic analysis of the NGS reads that were not removed in step (d).

In an aspect, this disclosure provides a method of sequencing comprising: (a) amplifying a population of distinct initial target DNA molecules from a tagged genomic sample thereby producing a population of amplified target DNA molecules, where the distinct initial target DNA molecules that comprise a polymorphic target sequence are tagged with different unique molecular identifier (UMI) sequences, where the UMI sequences comprise at least one nucleotide base selected from: R, Y, S, W, K, M, B, D, H, V, N and modified versions thereof, and where each of a plurality of the amplified target DNA molecules comprises the polymorphic target sequence and an associated UMI sequence of the different UMI sequences; (b) sequencing the plurality of the amplified target DNA molecules, thereby producing a plurality of NGS sequence reads, where the sequencing step provides, for each of the amplified target DNA molecules that are sequenced: the nucleotide sequence of: (i) at least a portion of the polymorphic target sequence; and (ii) an associated UMI sequence of the UMI sequences; (c) grouping the NGS reads into at least one UMI Family, where each NGS read within a UMI Family comprises an identical UMI sequence and aligns to the same polymorphic target sequence; (d) removing from consideration, for each polymorphic target sequence, all NGS reads in a below-threshold UMI Family; where the below-threshold UMI Family comprises a size smaller than X, where X is Y % of the mean value for the largest Z UMI Family sizes for the amplicon, where Y is between 1% and 20%, and where Z is between 1 and 20; and (e) generating a sequence variant call based on bioinformatic analysis of the NGS reads that were not removed in step (d).

In an aspect, this disclosure provides a method to analyze nucleic acid sequences, the method comprising: (a) attaching a unique molecular identifier (UMI) from a pool of UMIs to a first end of each strand of a plurality of analyte nucleic acid fragments to form a plurality of uniquely identified analyte nucleic acid fragments where the pool of UMIs is in excess of the plurality of analyte nucleic acid fragments; (b) redundantly determining nucleotide sequence of a uniquely identified analyte nucleic acid fragment, to generate next generation sequencing (NGS) reads where determined nucleotide sequences which share a UMI form a UMI Family; (c) removing from consideration, for each polymorphic target sequence, all NGS reads in a below-threshold UMI Family; where the below-threshold UMI Family comprises a size smaller than X, where X is Y % of the mean value for the largest Z UMI Family sizes for the amplicon, where Y is between 1% and 20%, and where Z is between 1 and 20; and (d) generating a sequence variant call based on bioinformatic analysis of the NGS reads that were not removed in step (c).

In an aspect, this disclosure provides a method for analyzing a DNA sample comprising at least one Target Region for potential sequence variants, the method comprising: (a) contacting the DNA sample with: (i) a set of unique molecular identifier (UMI) Primers, where each UMI primer comprises a UMI sequence and a gene-specific sequence that is complementary to a Target Region subsequence; (ii) a first DNA polymerase; and (iii) reagents and buffers needed for DNA polymerase extension to generate a mixture; (b) subjecting the mixture of step (a) to one or more temperatures that allow primer binding and DNA polymerase extension; (c) removing non-extended UMI primers to produce a product; (d) mixing the product of step (c) with: (i) a second set of DNA primers; (ii) a second DNA polymerase; and (iii) reagents and buffers needed for a polymerase chain reaction (PCR), and performing PCR to produce a PCR product; (e) subjecting the PCR product produced in step (d) to high-throughput DNA sequencing and obtaining a sequence file comprising next generation sequencing (NGS) reads; (0 grouping the NGS reads into at least a first UMI Family and a second UMI Family, where each NGS read within the first UMI Family comprises an identical UMI sequence and aligns to a common amplicon, where each NGS read within the second UMI Family comprises an identical UMI sequence and aligns to the common amplicon, and where the UMI sequence of the first UMI Family differs by 1 nucleotide or 2 nucleotides as compared to the UMI sequence of the second UMI Family; (g) removing from consideration the NGS reads in the UMI Family that has the fewest NGS reads between the first UMI Family and the second UMI Family; and (h) generating a sequence variant call based on bioinformatic analysis of the NGS reads that were not removed in step (g).

In an aspect, this disclosure provides a method for analyzing a DNA sample comprising at least one Target Region for potential sequence variants, the method comprising: (a) preparing a next generation sequencing (NGS) library, where a unique molecular identifier (UMI) sequence is added to a plurality of polynucleotides present in the NGS library; (b) obtaining a sequence file comprising NGS reads; (c) grouping the NGS reads into at least a first UMI Family and a second UMI Family, where each NGS read within the first UMI Family comprises an identical UMI sequence and aligns to a common amplicon, where each NGS read within the second UMI Family comprises an identical UMI sequence and aligns to the common amplicon, and where the UMI sequence of the first UMI Family differs by 1 nucleotide or 2 nucleotides as compared to the UMI sequence of the second UMI Family; (d) removing from consideration the NGS reads in the UMI Family that has the fewest NGS reads between the first UMI Family and the second UMI Family; and (e) generating a sequence variant call based on bioinformatic analysis of the NGS reads that were not removed in step (d).

In an aspect, this disclosure provides a method of sequencing, the method comprising: (a) amplifying a population of distinct initial target DNA molecules from a tagged genomic sample thereby producing a population of amplified target DNA molecules, where the distinct initial target DNA molecules that comprise a polymorphic target sequence are tagged with different unique molecular identifier (UMI) sequences, where the UMI sequences comprise at least one nucleotide base selected from: R, Y, S, W, K, M, B, D, H, V, N and modified versions thereof, and where each of a plurality of the amplified target DNA molecules comprises the polymorphic target sequence and an associated UMI sequence of the different UMI sequences; (b) sequencing the plurality of the amplified target DNA molecules, thereby producing a plurality of NGS sequence reads, where the sequencing step provides, for each of the amplified target DNA molecules that are sequenced: the nucleotide sequence of: (i) at least a portion of the polymorphic target sequence; and (ii) an associated UMI sequence of the UMI sequences; (c) grouping the NGS reads into at least a first UMI Family and a second UMI Family, where each NGS read within the first UMI Family comprises an identical UMI sequence and aligns to the polymorphic target sequence, where each NGS read within the second UMI Family comprises an identical UMI sequence and aligns to the polymorphic target sequence, and where the UMI sequence of the first UMI Family differs by 1 nucleotide or 2 nucleotides as compared to the UMI sequence of the second UMI Family; (d) removing from consideration the NGS reads in the UMI Family that has the fewest NGS reads between the first UMI Family and the second UMI Family; and (e) generating a sequence variant call based on bioinformatic analysis of the NGS reads that were not removed in step (d).

In an aspect, this disclosure provides a method to analyze nucleic acid sequences, the method comprising: (a) attaching a unique molecular identifier (UMI) from a pool of UMIs to a first end of each strand of a plurality of analyte nucleic acid fragments to form a plurality of uniquely identified analyte nucleic acid fragments where the pool of UMIs is in excess of the plurality of analyte nucleic acid fragments; (b) redundantly determining nucleotide sequence of a uniquely identified analyte nucleic acid fragment to generate next generation sequencing (NGS) reads, where determined nucleotide sequences which share a UMI form a UMI Family; (c) grouping the determined nucleotide sequences into at least a first UMI Family and a second UMI Family, where each determined nucleotide sequence within the first UMI Family comprises an identical UMI sequence and aligns to a common amplicon, where each determined nucleotide sequence within the second UMI Family comprises an identical UMI sequence and aligns to the common amplicon, and where the UMI sequence of the first UMI Family differs by 1 nucleotide or 2 nucleotides as compared to the UMI sequence of the second UMI Family; (d) removing from consideration the NGS reads in the UMI Family that has the fewest determined nucleotide sequences between the first UMI Family and the second UMI Family; and (e) generating a sequence variant call based on bioinformatic analysis of the remaining determined nucleotide sequences.

As used herein, “DNA” refers to deoxyribonucleic acid. DNA can be either single-stranded or double-stranded. DNA typically comprises four nucleotides: cytosine (C), guanine (G), adenine (A), and thymine (T). In an aspect, the sequence of a DNA molecule provided herein comprises one or more degenerate nucleotides. As used herein, a “degenerate nucleotide” refers to a nucleotide that can perform the same function or yield the same output as a structurally different nucleotide. Non-limiting examples of degenerate nucleotides include a C, G, or T nucleotide (B); an A, G, or T nucleotide (D); an A, C, or T nucleotide (H); a G or T nucleotide (K); an A or C nucleotide (M); any nucleotide (N); an A or G nucleotide (R); a G or C nucleotide (S); an A, C, or G nucleotide (V); an A or T nucleotide (W), and a C or T nucleotide (Y).

In an aspect, a UMI sequence comprises between 7 degenerate nucleotides and degenerate nucleotides. In an aspect, a UMI sequence comprises between 5 degenerate nucleotides and 40 degenerate nucleotides. In an aspect, a UMI sequence comprises between 10 degenerate nucleotides and 20 degenerate nucleotides. In an aspect, a UMI sequence comprises at least 5 degenerate nucleotides. In an aspect, a UMI sequence comprises at least 7 degenerate nucleotides. In an aspect, a UMI sequence comprises at least 10 degenerate nucleotides. In an aspect, a UMI sequence comprises at least 15 degenerate nucleotides. In an aspect, a UMI sequence comprises fewer than 50 degenerate nucleotides. In an aspect, a UMI sequence comprises fewer than 40 degenerate nucleotides. In an aspect, a UMI sequence comprises fewer than 30 degenerate nucleotides. In an aspect, a UMI sequence comprises fewer than 20 degenerate nucleotides.

In an aspect, each degenerate nucleotide in a UMI sequence is selected from the group consisting of N, B, D, H, V, S, W, Y, R, M, and K.

In an aspect, a UMI sequence comprises between 7 degenerate nucleotides and 30 degenerate nucleotides, where each degenerate nucleotide is selected from the group consisting of N, B, D, H, V, S, W, Y, R, M, and K.

In an aspect, a sequence variant call comprises removal of NGS reads when the UMI sequence of the NGS reads does not comprise an appropriate degenerate base design pattern. As used herein, an “appropriate degenerate base design pattern” refers to a UMI sequence comprising the expected number of degenerate bases and the expected type of degenerate bases for a given method. Non-limiting examples of inappropriate degenerate base designs would include UMI sequences comprising too many degenerate bases or too few degenerate bases.

As used herein, a “Target Region” refers to a DNA region of interest. In an aspect, a Target Region comprises a gene sequence. In an aspect, a Target Region comprises an exon sequence. In an aspect, a Target Region comprises an intron sequence. In an aspect, a Target Region comprises a 5′ untranslated region (UTR) sequence. In an aspect, a Target Region comprises a 3′ UTR sequence. In an aspect, a Target Region comprises at least 5 nucleotides. In an aspect, a Target Region comprises at least 25 nucleotides. In an aspect, a Target Region comprises at least 50 nucleotides. In an aspect, a Target Region comprises at least 100 nucleotides. In an aspect, a Target Region comprises at least 500 nucleotides. In an aspect, a Target Region comprises at least 1000 nucleotides. In an aspect, a Target Region comprises at least 5000 nucleotides. In an aspect, a Target Region comprises between 5 nucleotides and 10,000 nucleotides. In an aspect, a Target Region comprises between 5 nucleotides and 5,000 nucleotides. In an aspect, a Target Region comprises between 5 nucleotides and 1,000 nucleotides. In an aspect, a Target Region comprises between 5 nucleotides and 500 nucleotides. In an aspect, a Target Region comprises between 5 nucleotides and 100 nucleotides.

In an aspect, a DNA sample provided herein comprises between 1 Target Region and 10,000 Target Regions. In an aspect, a DNA sample provided herein comprises between 1 Target Region and 100,000 Target Regions. In an aspect, a DNA sample provided herein comprises between 1 Target Region and 1000 Target Regions. In an aspect, a DNA sample provided herein comprises between 1 Target Region and 500 Target Regions. In an aspect, a DNA sample provided herein comprises between 1 Target Region and 100 Target Regions. In an aspect, a DNA sample provided herein comprises between 1 Target Region and 10 Target Regions. In an aspect, a DNA sample provided herein comprises at least 1 Target Region. In an aspect, a DNA sample provided herein comprises at least 2 Target Regions. In an aspect, a DNA sample provided herein comprises at least 10 Target Regions. In an aspect, a DNA sample provided herein comprises at least 50 Target Regions. In an aspect, a DNA sample provided herein comprises at least 100 Target Regions. In an aspect, a DNA sample provided herein comprises at least 1000 Target Regions. In an aspect, a DNA sample provided herein comprises at least 10,000 Target Regions. In an aspect, a DNA sample provided herein comprises at least 100,000 Target Regions.

In an aspect, a Target Region comprises at least 1 sequence variant. In an aspect, a Target Region comprises at least 2 sequence variants. In an aspect, a Target Region comprises at least 5 sequence variants. In an aspect, a Target Region comprises at least 10 sequence variants. In an aspect, a Target Region comprises at least 20 sequence variants.

In an aspect, a sequence variant of a Target Region is present in a population at a frequency of at least 0.1%. In an aspect, a sequence variant of a Target Region is present in a population at a frequency of at least 0.25%. In an aspect, a sequence variant of a Target Region is present at a frequency of at least 0.5%. In an aspect, a sequence variant of a Target Region is present at a frequency of at least 0.75%. In an aspect, a sequence variant of a Target Region is present in a population at a frequency of at least 1%. In an aspect, a sequence variant of a Target Region is present in a population at a frequency of at least 1.5%. In an aspect, a sequence variant of a Target Region is present in a population at a frequency of at least 2%. In an aspect, a sequence variant of a Target Region is present in a population at a frequency of at least 2.5%. In an aspect, a sequence variant of a Target Region is present in a population at a frequency of at least 3%. In an aspect, a sequence variant of a Target Region is present in a population at a frequency of at least 4%. In an aspect, a sequence variant of a Target Region is present in a population at a frequency of at least 5%. In an aspect, a sequence variant of a Target Region is present in a population at a frequency of at least 6%. In an aspect, a sequence variant of a Target Region is present in a population at a frequency of at least 7%. In an aspect, a sequence variant of a Target Region is present in a population at a frequency of at least 8%. In an aspect, a sequence variant of a Target Region is present in a population at a frequency of at least 9%. In an aspect, a sequence variant of a Target Region is present in a population at a frequency of at least 10%. In an aspect, a sequence variant of a Target Region is present in a population at a frequency of between 0.1% and 10%. In an aspect, a sequence variant of a Target Region is present in a population at a frequency of between 0.1% and 7.5%. In an aspect, a sequence variant of a Target Region is present in a population at a frequency of between 0.1% and 5%. In an aspect, a sequence variant of a Target Region is present in a population at a frequency of between 0.1% and 2.5%. In an aspect, a sequence variant of a Target Region is present in a population at a frequency of between 0.1% and 1%. In an aspect, a sequence variant of a Target Region is present in a population at a frequency of between 0.5% and 5%. In an aspect, a sequence variant of a Target Region is present in a population at a frequency of between 0.5% and 2.5%. In an aspect, a sequence variant of a Target Region is present in a population at a frequency of between 2% and 5%.

As used herein, a “sequence variant,” refers to a change in at least one nucleotide in a sequence as compared to a reference, or “wildtype” sequence of a Target Region. As used herein, a “sequence variant call” refers to the identification of a sequence as comprising a sequence variant as compared to a wildtype sequence. As used herein, a “wildtype sequence” refers to the reference sequence for a given gene or amplicon. In an aspect, a sequence variant refers to an allele of a Target Region. As used herein, a “DNA variant molecule” refers to a DNA molecule comprising a sequence variant.

In an aspect, a sequence variant comprises a single nucleotide polymorphism (SNP). In an aspect, a sequence variant comprises an insertion of at least one nucleotide. In an aspect, a sequence variant comprises a deletion of at least one nucleotide. In an aspect, a sequence variant comprises an inversion of at least two nucleotides.

In an aspect, a reference sequence of at least one Target Region comprises multiple DNA sequences for each Target Region comprising single nucleotide polymorphism alleles comprising a population allele frequency of greater than 0.1%. In an aspect, a reference sequence of at least one Target Region comprises multiple DNA sequences for each Target Region comprising single nucleotide polymorphism alleles comprising a population allele frequency of greater than 0.25%. In an aspect, a reference sequence of at least one Target Region comprises multiple DNA sequences for each Target Region comprising single nucleotide polymorphism alleles comprising a population allele frequency of greater than 0.5%. In an aspect, a reference sequence of at least one Target Region comprises multiple DNA sequences for each Target Region comprising single nucleotide polymorphism alleles comprising a population allele frequency of greater than 1%. In an aspect, a reference sequence of at least one Target Region comprises multiple DNA sequences for each Target Region comprising single nucleotide polymorphism alleles comprising a population allele frequency of greater than 1.5%. In an aspect, a reference sequence of at least one Target Region comprises multiple DNA sequences for each Target Region comprising single nucleotide polymorphism alleles comprising a population allele frequency of greater than 2%. In an aspect, a reference sequence of at least one Target Region comprises multiple DNA sequences for each Target Region comprising single nucleotide polymorphism alleles comprising a population allele frequency of between 0.1% and 5%. In an aspect, a reference sequence of at least one Target Region comprises multiple DNA sequences for each Target Region comprising single nucleotide polymorphism alleles comprising a population allele frequency of between 0.1% and 2.5%.

In an aspect, this disclosure provides unique molecular identifiers (UMIs). As used herein, a “unique molecular identifier” refers to a unique nucleotide sequence that serves as a molecular barcode for an individual molecule. UMIs are often attached to DNA molecules in a sample library to uniquely tag each molecule. UMIs enable error correction and increased accuracy during sequencing of DNA molecules.

As used herein, a “UMI Family” refers to a group of NGS reads that comprise identical UMI sequences and also aligns to the same amplicon. In an aspect, a UMI Family comprises at least 1 NGS read. In an aspect, a UMI Family comprises at least 2 NGS reads. In an aspect, a UMI Family comprises at least 5 NGS reads. In an aspect, a UMI Family comprises at least 10 NGS reads. In an aspect, a UMI Family comprises at least 50 NGS reads. In an aspect, a UMI Family comprises at least 100 NGS reads. In an aspect, a UMI Family comprises at least 500 NGS reads. In an aspect, a UMI Family comprises at least 1000 NGS reads. In an aspect, a UMI Family comprises at least 2500 NGS reads. In an aspect, a UMI Family comprises between 1 NGS read and 10,000 NGS reads. In an aspect, a UMI Family comprises between 1 NGS read and 5,000 NGS reads. In an aspect, a UMI Family comprises between 1 NGS read and 1000 NGS reads. In an aspect, a UMI Family comprises between 1 NGS read and 100 NGS reads.

In an aspect, a sequence variant call comprises identifying a UMI Family Sequence. As used herein, a “UMI Family Sequence” refers to the most frequent nucleotide sequence within a UMI Family.

In an aspect, a sequence variant call comprises the removal of NGS reads when between 1 NGS read and 100 NGS reads comprise an identical UMI sequence. In an aspect, a sequence variant call comprises the removal of NGS reads when between 1 NGS read rand 10 NGS reads comprise an identical UMI sequence. In an aspect, a sequence variant call comprises the removal of NGS reads when between 1 NGS read and 1000 NGS reads comprise an identical UMI sequence. In an aspect, a sequence variant call comprises the removal of NGS reads when between 2 NGS reads and 100 NGS reads comprise an identical UMI sequence. In an aspect, a sequence variant call comprises the removal of NGS reads when between 2 NGS reads and 10 NGS reads comprise an identical UMI sequence. In an aspect, a sequence variant call comprises the removal of NGS reads when between 2 NGS reads and 1000 NGS reads comprise an identical UMI sequence.

In an aspect, a sequence variant call comprises the removal of NGS reads when at least 2 NGS reads comprise an identical UMI sequence. In an aspect, a sequence variant call comprises the removal of NGS reads when at least 10 NGS reads comprise an identical UMI sequence. In an aspect, a sequence variant call comprises the removal of NGS reads when at least 50 NGS reads comprise an identical UMI sequence.

As used herein, an “amplicon” refers to a copy of DNA made via PCR.

In an aspect, this disclosure provides UMI Primers. As used herein, a “UMI Primer” is an oligonucleotide molecule comprising a UMI sequence and a gene-specific sequence that is complementary to a Target Region subsequence. In an aspect, a gene-specific sequence is 100% complementary to a Target Region subsequence. In an aspect, a gene-specific sequence is at least 99% complementary to a Target Region subsequence. In an aspect, a gene-specific sequence is at least 98% complementary to a Target Region subsequence. In an aspect, a gene-specific sequence is at least 97% complementary to a Target Region subsequence. In an aspect, a gene-specific sequence is at least 96% complementary to a Target Region subsequence. In an aspect, a gene-specific sequence is at least 95% complementary to a Target Region subsequence. In an aspect, a gene-specific sequence is at least 90% complementary to a Target Region subsequence. In an aspect, a gene-specific sequence is at least 85% complementary to a Target Region subsequence. In an aspect, a gene-specific sequence is at least 80% complementary to a Target Region subsequence.

As used herein, a “Target Region subsequence” comprises at least 1 fewer nucleotides as compared to a full-length Target Region. In an aspect, a Target Region subsequence comprises at least 5 nucleotides. In an aspect, a Target Region subsequence comprises at least 15 nucleotides. In an aspect, a Target Region subsequence comprises at least 25 nucleotides. In an aspect, a Target Region subsequence comprises at least 35 nucleotides. In an aspect, a Target Region subsequence comprises at least 50 nucleotides. In an aspect, a Target Region subsequence comprises at least 75 nucleotides. In an aspect, a Target Region subsequence comprises at least 100 nucleotides. In an aspect, a Target Region subsequence comprises between 5 and 500 nucleotides. In an aspect, a Target Region subsequence comprises between 5 and 250 nucleotides. In an aspect, a Target Region subsequence comprises between 5 and 100 nucleotides. In an aspect, a Target Region subsequence comprises between 5 and 50 nucleotides. In an aspect, a Target Region subsequence comprises between 5 and 35 nucleotides. In an aspect, a Target Region subsequence comprises between 15 and 35 nucleotides.

In an aspect, non-extended UMI primers are removed from a mixture via a method selected from the group consisting of solid phase reversible immobilization purification, column purification, and enzymatic digestion. In an aspect, non-extended UMI primers are removed from a mixture via solid phase reversible immobilization purification. In an aspect, non-extended UMI primers are removed from a mixture via column purification. In an aspect, non-extended UMI primers are removed from a mixture via enzymatic digestion.

In an aspect, a UMI Primer comprises, in order from 5′ to 3′, (a) a first universal region; (b) an optional second region comprising a length of between 1 nucleotide and 50 nucleotides; (c) a third region comprising a UMI sequence; and (d) a fourth region comprising a gene-specific sequence that is complementary to a Target Region subsequence. As used herein, a “universal region” refers to sequences that remain the same in UMI primers designed for different Target Regions.

In an aspect, a method comprises the introduction of a set of Outer Primers and a set of Inner Primers, where between 3 nucleotides and 20 nucleotides positioned at the 3′ end of the Inner Primer are not subsequences of the set of Outer Primers. As used herein, “Outer Primers” refers to primers that flank a set of “Inner Primers” on a Target Region. For example, without being limiting, a first (e.g., forward) Outer Primer is positioned 5′ to a first (e.g., forward) Inner Primer and a second (e.g., reverse) Outer Primer is positioned 3′ to a second (e.g., reverse) Inner Primer.

In an aspect, this disclosure provides at least one DNA polymerase. As used herein, a “DNA polymerase” refers to an enzyme that is capable of catalyzing the synthesis of a DNA molecule from nucleoside triphosphates. DNA polymerases add a nucleotide to the 3′ end of a DNA strand one nucleotide at a time, creating an antiparallel DNA strand as compared to a template DNA strand. DNA polymerases are unable to begin a new DNA molecule de novo; they require a primer to which it can add a first new nucleotide.

In an aspect, this disclosure provides reagents and buffers needed for DNA polymerase extension. Non-limiting examples of reagents and buffers needed for DNA polymerase extension include Tris-HCl, potassium chloride, magnesium chloride, oligonucleotide primers, deoxynucleotides (dNTPs), betaine, and dimethyl sulfoxide. Those of ordinary skill in the art recognize that different DNA polymerases and different Target Regions can require different groupings of necessary reagents and buffers.

DNA polymerases can extend primers at different temperatures, depending on the DNA polymerase. In an aspect, a DNA polymerase extends primers at a temperature of at least 40° C. In an aspect, a DNA polymerase extends primers at a temperature of at least In an aspect, a DNA polymerase extends primers at a temperature of at least 55° C. In an aspect, a DNA polymerase extends primers at a temperature of at least 60° C. In an aspect, a DNA polymerase extends primers at a temperature of at least 65° C. In an aspect, a DNA polymerase extends primers at a temperature of at least 70° C. In an aspect, a DNA polymerase extends primers at a temperature of at least 75° C. In an aspect, a DNA polymerase extends primers at a temperature of at least 80° C.

Primers can bind, or anneal, to a complementary part of a Target Region at a variety of temperatures, depending on the structure and length of the sequences involved. In an aspect, primer binding occurs at a temperature of at least 35° C. In an aspect, primer binding occurs at a temperature of at least 40° C. In an aspect, primer binding occurs at a temperature of at least 45° C. In an aspect, primer binding occurs at a temperature of at least In an aspect, primer binding occurs at a temperature of at least 55° C. In an aspect, primer binding occurs at a temperature of at least 60° C. In an aspect, primer binding occurs at a temperature of at least 65° C. In an aspect, primer binding occurs at a temperature of at least 70° C.

In an aspect, DNA polymerase extension and primer binding occur at different temperatures. In an aspect, DNA polymerase extension and primer binding occur at the same temperature.

In an aspect, a DNA polymerase is a thermostable DNA polymerase. As used herein, a “thermostable DNA polymerase” refers to DNA polymerases that can function at high temperatures (e.g., greater than 65° C.) and can survive higher temperatures (e.g., up to about 100° C.). Thermostable DNA polymerases often have maximal catalytic activity at temperatures between 70° C. and 80° C. In an aspect, a thermostable DNA polymerase is selected from the group consisting of comprising Taq DNA polymerase, Phusion® DNA polymerase, Q5C) DNA polymerase, and KAPA High Fidelity DNA polymerase.

In an aspect, a DNA polymerase is a non-thermostable DNA polymerase. As used herein, a “non-thermostable DNA polymerase” refers to DNA polymerases that cannot function at high temperatures. In an aspect, a non-thermostable DNA polymerase is selected from the group consisting of phi29 DNA polymerase and Bst DNA polymerase.

In an aspect, a method comprises high-throughput sequencing. In an aspect, a method comprises subjecting a plurality of amplicons to high-throughput sequencing. As used herein, “high-throughput sequencing” refers to any sequences method that is capable of sequencing multiple (e.g., tens, hundreds, thousands, millions, hundreds of millions) DNA molecules in parallel. In an aspect, Sanger sequencing is not high-throughput sequencing. In an aspect, high-throughput sequencing comprises the use of a sequencing-by-synthesis (SBS) flow cell. In an aspect, an SBS flow cell is selected from the group consisting of an Illumina SBS flow cell and a Pacific Biosciences (PacBio) SBS flow cell. In an aspect, high-throughput sequencing is performed via electrical current measurements in conjunction with an Oxford nanopore.

In an aspect, high-throughput DNA sequencing comprises sequencing-by-synthesis or nanopore-based sequencing.

Typically, high-throughput sequencing generates a sequence file. As used herein, a “sequence file” refers to a computer-readable text file that comprises the sequence of at least one next generation sequencing (NGS) read. As used herein, an “NGS read” refers to a nucleotide sequence of a single nucleic acid molecule generated via a high-throughput sequencing method. In an aspect, an NGS read comprises a UMI sequence. In an aspect, an NGS read comprises a gene sequence. In an aspect, an NGS read comprises a UMI sequence and a gene sequence. In an aspect, an NGS read comprises at least 10 nucleotides. In an aspect, an NGS read comprises at least 25 nucleotides. In an aspect, an NGS read comprises at least 50 nucleotides. In an aspect, an NGS read comprises at least 100 nucleotides. In an aspect, an NGS read comprises at least 250 nucleotides. In an aspect, an NGS read comprises at least 500 nucleotides. In an aspect, an NGS read comprises at least 1000 nucleotides. In an aspect, an NGS read comprises between 10 nucleotides and 10,000 nucleotides. In an aspect, an NGS read comprises between 10 nucleotides and 1000 nucleotides. In an aspect, an NGS read comprises between 25 nucleotides and 150 nucleotides.

In an aspect, a sequence file is plain sequence format. In an aspect, a sequence file is in FASTQ format. In an aspect, a sequence file is in EMBL format. In an aspect, a sequence file is in FASTA format. In an aspect, a sequence file is in GCG format. In an aspect, a sequence file is in GCG-rich sequence format. In an aspect, a sequence file is in GenBank format. In an aspect, a sequence file is in IG format.

In an aspect, an identified NGS sequence comprises a vetoed UMI sequence. As used herein, a “vetoed UMI sequence” refers to the UMI sequence of a NGS read that comprises a gene sequence identical to a wildtype sequence of at least one Target Region. If the number of NGS reads comprising the vetoed UMI sequence and a wildtype sequence passes a threshold, any NGS reads comprising the vetoed UMI sequence (regardless of gene sequence) are removed from sequence variant analysis.

As used herein, a “tagged” genomic sample or nucleic acid molecule refers to a genome sample or nucleic acid molecule comprising at least one UMI sequence.

As used herein, a “polymorphic target sequence” is a sequence that comprises one or more sequence variants in a given population. In contrast, an “invariant target sequence” does not comprise any sequence variants in a given population.

In an aspect, a method comprises removing from consideration, for each amplicon, all NGS reads in a below-threshold UMI Family. As used herein, a “below-threshold UMI Family” refers to a UMI Family that comprises fewer than X NGS reads, where X is determined as Y % of the mean value for the largest Z UMI Family sizes for a given amplicon. In an aspect, Y is between 1% and 20% and Z is between 1 and 20. In an aspect, Y is between 1% and 50% and Z is between 1 and 50. In an aspect, Y is between 1% and 75% and Z is between 1 and 75. In an aspect, Y is greater than 1% and Z is greater than 1. In an aspect, Y is greater than 5% and Z is greater than 5. In an aspect, Y is greater than 10% and Z is greater than 10. In an aspect, Y and Z are the same integer. In an aspect, Y and Z are different integers. In an aspect, X and Y are the same integer. In an aspect, X and Y are different integers. In an aspect X and Z are the same integer. In an aspect, X and Z are different integers. In an aspect, X, Y, and Z are the same integer. In an aspect, X, Y, and Z are different integers.

In an aspect, a sequence variant call comprises removing from consideration, for each amplicon, all NGS reads in a below-threshold UMI Family, where the below-threshold UMI Family comprises a size smaller than X, where X is Y % of the mean value for the largest Z UMI Family sizes for the amplicon. In an aspect, Y is between 1% and 20% and Z is between 1 and 20. In an aspect, Y is between 1% and 50% and Z is between 1 and 50. In an aspect, Y is between 1% and 75% and Z is between 1 and 75. In an aspect, Y is greater than 1% and Z is greater than 1. In an aspect, Y is greater than 5% and Z is greater than 5. In an aspect, Y is greater than 10% and Z is greater than 10. In an aspect, Y and Z are the same integer. In an aspect, Y and Z are different integers. In an aspect, X and Y are the same integer. In an aspect, X and Y are different integers. In an aspect X and Z are the same integer. In an aspect, X and Z are different integers. In an aspect, X, Y, and Z are the same integer. In an aspect, X, Y, and Z are different integers.

In an aspect, a sequence variant call comprises removal of at least one UMI Family comprising a member size smaller than X for a given amplicon, where X is set as Y % of the mean value for the largest Z UMI Family size(s) for the amplicon. In an aspect, Y is between 1% and 20% and Z is between 1 and 20. In an aspect, Y is between 1% and 50% and Z is between 1 and 50. In an aspect, Y is between 1% and 75% and Z is between 1 and 75. In an aspect, Y is greater than 1% and Z is greater than 1. In an aspect, Y is greater than 5% and Z is greater than 5. In an aspect, Y is greater than 10% and Z is greater than In an aspect, Y and Z are the same integer. In an aspect, Y and Z are different integers. In an aspect, X and Y are the same integer. In an aspect, X and Y are different integers. In an aspect X and Z are the same integer. In an aspect, X and Z are different integers. In an aspect, X, Y, and Z are the same integer. In an aspect, X, Y, and Z are different integers.

In an aspect, a first UMI Family and a second UMI family comprise different UMI sequences, but both align to a common amplicon. In an aspect, the UMI sequence of a first UMI Family differs from the UMI sequence of a second UMI Family by one nucleotide. In an aspect, the UMI sequence of a first UMI Family differs from the UMI sequence of a second UMI Family by two nucleotides. In an aspect, the UMI sequence of a first UMI Family differs from the UMI sequence of a second UMI Family by three nucleotides. In an aspect, the UMI sequence of a first UMI Family differs from the UMI sequence of a second UMI Family by four nucleotides. In an aspect, the UMI sequence of a first UMI Family differs from the UMI sequence of a second UMI Family by five nucleotides. In an aspect, the UMI sequence of a first UMI Family differs from the UMI sequence of a second UMI Family by one nucleotide or two nucleotides. In an aspect, the UMI sequence of a first UMI Family differs from the UMI sequence of a second UMI Family by between one nucleotide and three nucleotides.

As a non-limiting example, the sequence 5′-AATG-3′ differs from the sequence by one nucleotide. As a non-limiting example, the sequence 5′-AATG-3′ differs from the sequence 5′-AAAC-3′ by two nucleotides.

In an aspect, a sequence variant call comprises (a) grouping NGS reads into at least a first UMI Family and a second UMI Family, where each NGS read within the first UMI Family comprises a first identical UMI sequence and aligns to a common amplicon, where each NGS read within the second UMI Family comprises a second identical UMI sequence and aligns to the same common amplicon, and where the UMI sequence of the first UMI Family differs by 1 nucleotide or 2 nucleotides as compared to the UMI sequence of the second UMI Family; and (b) removing from consideration the NGS reads in the UMI Family that has the fewest NGS reads between the first UMI Family and the second UMI Family.

In an aspect, a sequence variant call comprises identifying one or more UMI Families comprising between 1 NGS and 10 NGS reads comprising a sequence 100% identical to a reference sequence of a Target Region. In an aspect, a sequence variant call comprises identifying one or more UMI Families comprising between 1 NGS and 50 NGS reads comprising a sequence 100% identical to a reference sequence of a Target Region. In an aspect, a sequence variant call comprises identifying one or more UMI Families comprising between 1 NGS and 100 NGS reads comprising a sequence 100% identical to a reference sequence of a Target Region. In an aspect, a sequence variant call comprises identifying one or more UMI Families comprising between 1 NGS and 1000 NGS reads comprising a sequence 100% identical to a reference sequence of a Target Region. In an aspect, a sequence variant call comprises identifying one or more UMI Families comprising at least 1 NGS read comprising a sequence 100% identical to a reference sequence of a Target Region. In an aspect, a sequence variant call comprises identifying one or more UMI Families comprising at least 5 NGS reads comprising a sequence 100% identical to a reference sequence of a Target Region. In an aspect, a sequence variant call comprises identifying one or more UMI Families comprising at least 10 NGS reads comprising a sequence 100% identical to a reference sequence of a Target Region.

In an aspect, a method comprises variant sequence enrichment. As used herein, “variant sequence enrichment” refers to a protocol that enhances the ability to detect rare (e.g., occurring at a frequency of less than 5% in a given population) sequence variants for a Target Region. In an aspect, variant sequence enrichment is performed by blocker displacement amplification (BDA). See, for example, WO 2019/164885, which is incorporated herein by reference in its entirety. In an aspect, BDA comprises amplifying a nucleic acid molecule with: (a) a BDA forward primer for each target genomic region, where the BDA forward primer comprises a region targeting a specific genomic region; and (b) a BDA blocker for each target genomic region, where 4 or more nucleotides at the 3′ end of the BDA forward primer sequence are also present at or near the 5′ end of the BDA blocker sequence, and where the BDA blocker comprises a 3′ sequence or modification that prevents extension by the DNA polymerase, and where the concentration of the BDA blocker is at least twice the concentration of the BDA forward primer.

The following exemplary, non-limiting, embodiments are envisioned:
1. A method for analyzing a DNA sample comprising at least one Target Region for potential sequence variants, the method comprising:
   (a) contacting the DNA sample with:
      (i) a set of unique molecular identifier (UMI) Primers, where each UMI Primer comprises a UMI sequence and a gene-specific sequence that is complementary to a Target Region subsequence;
      (ii) a first DNA polymerase; and
      (iii) reagents and buffers needed for DNA polymerase extension to generate a mixture;
   (b) subjecting the mixture of step (a) to one or more temperatures that allow primer binding and DNA polymerase extension;
   (c) removing non-extended UMI Primers to produce a product;
   (d) mixing the product of step (c) with:
      (i) a second set of DNA primers;
      (ii) a second DNA polymerase; and
      (iii) reagents and buffers needed for a polymerase chain reaction (PCR),
   and performing PCR to produce a PCR product;
   (e) subjecting the PCR product produced in step (d) to high-throughput DNA sequencing and obtaining a sequence file comprising next generation sequencing (NGS) reads;
   (f) identifying a vetoed UMI sequence, where at least 1, 2, 3, 4, 5, 6, 7, 8, 9, or 10 NGS reads containing the vetoed UMI sequence also comprise a wildtype sequence of the at least one Target Region;
   (g) removing from consideration all NGS reads comprising the vetoed UMI sequence identified in step (0; and
   (h) generating a sequence variant call by quantifying DNA variant molecules based on bioinformatic analysis of the NGS reads that are not removed in step (g).
2. A method for analyzing a DNA sample comprising at least one Target Region for potential sequence variants, the method comprising:
   (a) preparing a next generation sequencing (NGS) library, where a unique molecular identifier (UMI) sequence is added to a plurality of polynucleotides present in the NGS library;
   (b) obtaining a sequence file comprising NGS reads;
   (c) identifying a vetoed UMI sequence, where at least 1, 2, 3, 4, 5, 6, 7, 8, 9, or 10 NGS reads containing the vetoed UMI sequence also comprise a WT sequence of the at least one Target Region;
   (d) removing from consideration all NGS reads comprising the vetoed UMI sequence identified in step (c); and
   (e) generating a sequence variant call by quantifying DNA variant molecules based on bioinformatic analysis of the NGS reads that are not removed in step (d).
3. A method of sequencing, comprising:
   (a) amplifying a population of distinct initial target DNA molecules from a tagged genomic sample thereby producing a population of amplified target DNA molecules, where the distinct initial target DNA molecules that comprise a polymorphic target sequence are tagged with different unique molecular identifier (UMI) sequences, where the UMI sequences comprise at least one nucleotide base selected from: R, Y, S, W, K, M, B, D, H, V, N and modified versions thereof, and where each of a plurality of the amplified target DNA molecules comprises the polymorphic target sequence and an associated UMI sequence of the different UMI sequences;
   (b) sequencing the plurality of the amplified target DNA molecules, thereby producing a plurality of NGS sequence reads, where the sequencing step provides, for each of the amplified target DNA molecules that are sequenced: the nucleotide sequence of: (i) at least a portion of the polymorphic target sequence; and (ii) an associated UMI sequence of the UMI sequences;
   (c) identifying a vetoed UMI sequence, where at least 1, 2, 3, 4, 5, 6, 7, 8, 9, or 10 NGS reads containing the vetoed UMI sequence also comprise a WT sequence of the at least one Target Region;
   (d) removing from consideration all NGS reads comprising the vetoed UMI sequence identified in step (c); and
   (e) generating a sequence variant call by quantifying DNA variant molecules based on bioinformatic analysis of the NGS reads that are not removed in step (d).
4. A method to analyze nucleic acid sequences, the method comprising:
   (a) attaching a unique molecular identifier (UMI) from a pool of UMIs to a first end of each strand of a plurality of analyte nucleic acid fragments to form a plurality of uniquely identified analyte nucleic acid fragments where the pool of UMIs is in excess of the plurality of analyte nucleic acid fragments;
   (b) redundantly determining nucleotide sequence of a uniquely identified analyte nucleic acid fragment to generate next generation sequencing (NGS) reads, where determined nucleotide sequences which share a UMI form a UMI Family;
   (c) identifying a vetoed UMI sequence, where at least 1, 2, 3, 4, 5, 6, 7, 8, 9, or 10 NGS reads containing the vetoed UMI sequence also comprise a WT sequence of the at least one Target Region;
   (d) removing from consideration all NGS reads comprising the vetoed UMI sequence identified in step (c); and
   (e) generating a sequence variant call by quantifying DNA variant molecules based on bioinformatic analysis of the NGS reads that are not removed in step (d).
5. A method for analyzing a DNA sample comprising at least one Target Region for potential sequence variants, the method comprising:
   (a) contacting the DNA sample with:
      (i) a set of unique molecular identifier (UMI) Primers, where each UMI Primer comprises a UMI sequence and a gene-specific sequence that is complementary to a Target Region subsequence;
      (ii) a first DNA polymerase; and
      (iii) reagents and buffers needed for DNA polymerase extension to generate a mixture;
   (b) subjecting the mixture of step (a) to temperatures that allow primer binding and DNA polymerase extension;
   (c) removing non-extended UMI Primers to produce a product;
   (d) mixing the product of (c) with:
      (i) a second set of DNA primers;
      (ii) a second DNA polymerase; and
      (iii) reagents and buffers needed for a polymerase chain reaction (PCR),
   and performing PCR to produce a PCR product;
   (e) subjecting the PCR product produced in step (d) to high-throughput DNA sequencing and obtaining a sequence file comprising next generation sequencing (NGS) reads;
   (f) grouping the NGS reads into at least one UMI Family, where each NGS read within a UMI Family comprises an identical UMI sequence and aligns to the same amplicon;
   (g) removing from consideration, for each amplicon, all NGS reads in a below-threshold UMI Family; where the below-threshold UMI Family comprises a size smaller than X, where X is Y % of the mean value for the largest Z UMI Family sizes for the amplicon, where Y is between 1% and 20%, and where Z is between 1 and 20; and
   (h) generating a sequence variant call based on bioinformatic analysis of the NGS reads that were not removed in step (g).
6. A method for analyzing a DNA sample comprising at least one Target Region for potential sequence variants, the method comprising:
   (a) preparing a next generation sequencing (NGS) library, where a unique molecular identifier (UMI) sequence is added to a plurality of polynucleotides present in the NGS library;
   (b) obtaining a sequence file comprising NGS reads;
   (c) grouping the NGS reads into at least one UMI Family, where each NGS read within a UMI Family comprises an identical UMI sequence and aligns to the same amplicon;
   (d) removing from consideration, for each amplicon, all NGS reads in a below-threshold UMI Family; where the below-threshold UMI Family comprises a size smaller than X, where X is Y % of the mean value for the largest Z UMI Family sizes for the amplicon, where Y is between 1% and 20%, and where Z is between 1 and 20; and
   (e) generating a sequence variant call based on bioinformatic analysis of the NGS reads that were not removed in step (d).
7. A method of sequencing, comprising:
   (a) amplifying a population of distinct initial target DNA molecules from a tagged genomic sample thereby producing a population of amplified target DNA molecules, where the distinct initial target DNA molecules that comprise a polymorphic target sequence are tagged with different unique molecular identifier (UMI) sequences, where the UMI sequences comprise at least one nucleotide base selected from: R, Y, S, W, K, M, B, D, H, V, N and modified versions thereof, and where each of a plurality of the amplified target DNA molecules comprises the polymorphic target sequence and an associated UMI sequence of the different UMI sequences;
   (b) sequencing the plurality of the amplified target DNA molecules, thereby producing a plurality of NGS sequence reads, where the sequencing step provides, for each of the amplified target DNA molecules that are sequenced: the nucleotide sequence of: (i) at least a portion of the polymorphic target sequence; and (ii) an associated UMI sequence of the UMI sequences;
   (c) grouping the NGS reads into at least one UMI Family, where each NGS read within a UMI Family comprises an identical UMI sequence and aligns to the same polymorphic target sequence;
   (d) removing from consideration, for each polymorphic target sequence, all NGS reads in a below-threshold UMI Family; where the below-threshold UMI Family comprises a size smaller than X, where X is Y % of the mean value for the largest Z UMI Family sizes for the amplicon, where Y is between 1% and 20%, and where Z is between 1 and 20; and
   (e) generating a sequence variant call based on bioinformatic analysis of the NGS reads that were not removed in step (d).
8. A method to analyze nucleic acid sequences, the method comprising:
   (a) attaching a unique molecular identifier (UMI) from a pool of UMIs to a first end of each strand of a plurality of analyte nucleic acid fragments to form a plurality of uniquely identified analyte nucleic acid fragments where the pool of UMIs is in excess of the plurality of analyte nucleic acid fragments;
   (b) redundantly determining nucleotide sequence of a uniquely identified analyte nucleic acid fragment, to generate next generation sequencing (NGS) reads where determined nucleotide sequences which share a UMI form a UMI Family;
   (c) removing from consideration, for each polymorphic target sequence, all NGS reads in a below-threshold UMI Family; where the below-threshold UMI Family comprises a size smaller than X, where X is Y % of the mean value for the largest Z UMI Family sizes for the amplicon, where Y is between 1% and 20%, and where Z is between 1 and 20; and
   (d) generating a sequence variant call based on bioinformatic analysis of the NGS reads that were not removed in step (c).
9. A method for analyzing a DNA sample comprising at least one Target Region for potential sequence variants, the method comprising:
   (a) contacting the DNA sample with:
      (i) a set of unique molecular identifier (UMI) Primers, where each UMI Primer comprises a UMI sequence and a gene-specific sequence that is complementary to a Target Region subsequence;
      (ii) a first DNA polymerase; and
      (iii) reagents and buffers needed for DNA polymerase extension to generate a mixture;
   (b) subjecting the mixture of step (a) to temperatures that allow primer binding and DNA polymerase extension;
   (c) removing non-extended UMI Primers to produce a product;
   (d) mixing the product of (c) with:
      (i) a second set of DNA primers;
      (ii) a second DNA polymerase; and
      (iii) reagents and buffers needed for a polymerase chain reaction (PCR),
   and performing PCR to produce a PCR product;
   (e) subjecting the PCR product produced in step (d) to high-throughput DNA sequencing and obtaining a sequence file comprising next generation sequencing (NGS) reads;
   (f) grouping the NGS reads into at least a first UMI Family and a second UMI Family, where each NGS read within the first UMI Family comprises a first identical UMI sequence and aligns to a common amplicon, where each NGS read within the second UMI Family comprises a second identical UMI sequence and aligns to the common amplicon, and where the UMI sequence of the first UMI Family differs by 1 nucleotide or 2 nucleotides as compared to the UMI sequence of the second UMI Family;
   (g) removing from consideration the NGS reads in the UMI Family that has the fewest NGS reads between the first UMI Family and the second UMI Family; and
   (h) generating a sequence variant call based on bioinformatic analysis of the NGS reads that were not removed in step (g).
10. A method for analyzing a DNA sample comprising at least one Target Region for potential sequence variants, the method comprising:
   (a) preparing a next generation sequencing (NGS) library, where a unique molecular identifier (UMI) sequence is added to a plurality of polynucleotides present in the NGS library;
   (b) obtaining a sequence file comprising NGS reads;
   (c) grouping the NGS reads into at least a first UMI Family and a second UMI Family, where each NGS read within the first UMI Family comprises a first identical UMI sequence and aligns to a common amplicon, where each NGS read within the second UMI Family comprises a second identical UMI sequence and aligns to the common amplicon, and where the UMI sequence of the first UMI Family differs by 1 nucleotide or 2 nucleotides as compared to the UMI sequence of the second UMI Family;
   (d) removing from consideration the NGS reads in the UMI Family that has the fewest NGS reads between the first UMI Family and the second UMI Family; and
   (e) generating a sequence variant call based on bioinformatic analysis of the NGS reads that were not removed in step (d).
11. A method of sequencing, the method comprising:
   (a) amplifying a population of distinct initial target DNA molecules from a tagged genomic sample thereby producing a population of amplified target DNA molecules, where the distinct initial target DNA molecules that comprise a polymorphic target sequence are tagged with different unique molecular identifier (UMI) sequences, where the UMI sequences comprise at least one nucleotide base selected from: R, Y, S, W, K, M, B, D, H, V, N and modified versions thereof, and where each of a plurality of the amplified target DNA molecules comprises the polymorphic target sequence and an associated UMI sequence of the different UMI sequences;
   (b) sequencing the plurality of the amplified target DNA molecules, thereby producing a plurality of NGS sequence reads, where the sequencing step provides, for each of the amplified target DNA molecules that are sequenced: the nucleotide sequence of: (i) at least a portion of the polymorphic target sequence; and (ii) an associated UMI sequence of the UMI sequences;
   (c) grouping the NGS reads into at least a first UMI Family and a second UMI Family, where each NGS read within the first UMI Family comprises a first identical UMI sequence and aligns to the polymorphic target sequence, where each NGS read within the second UMI Family comprises a second identical UMI sequence and aligns to the polymorphic target sequence, and where the UMI sequence of the first UMI Family differs by 1 nucleotide or 2 nucleotides as compared to the UMI sequence of the second UMI Family;
   (d) removing from consideration the NGS reads in the UMI Family that has the fewest NGS reads between the first UMI Family and the second UMI Family; and
   (e) generating a sequence variant call based on bioinformatic analysis of the NGS reads that were not removed in step (d).
12. A method to analyze nucleic acid sequences, the method comprising:
   (a) attaching a unique molecular identifier (UMI) from a pool of UMIs to a first end of each strand of a plurality of analyte nucleic acid fragments to form a plurality of uniquely identified analyte nucleic acid fragments where the pool of UMIs is in excess of the plurality of analyte nucleic acid fragments;
   (b) redundantly determining nucleotide sequence of a uniquely identified analyte nucleic acid fragment to generate next generation sequencing (NGS) reads, where determined nucleotide sequences which share a UMI form a UMI Family;
   (c) grouping the determined nucleotide sequences into at least a first UMI Family and a second UMI Family, where each determined nucleotide sequence within the first UMI Family comprises a first identical UMI sequence and aligns to a common amplicon, where each determined nucleotide sequence within the second UMI Family comprises a second identical UMI sequence and aligns to the common amplicon, and where the UMI sequence of the first UMI Family differs by 1 nucleotide or 2 nucleotides as compared to the UMI sequence of the second UMI Family;
   (d) removing from consideration the NGS reads in the UMI Family that has the fewest determined nucleotide sequences between the first UMI Family and the second UMI Family; and
   (e) generating a sequence variant call based on bioinformatic analysis of the remaining determined nucleotide sequences.
13. The method of any one of embodiments 1, 2, 4-6, 8-10, or 12, where the UMI sequence comprises between 7 degenerate nucleotides and 30 degenerate nucleotides, and where each degenerate nucleotide is selected from the group consisting of N, B, D, H, V, S, W, Y, R, M, and K.
14. The method of any one of embodiments 1, 5, or 9, where the high-throughput DNA sequencing comprises sequencing-by-synthesis or nanopore-based sequencing.
15. The method of any one of embodiments 1, 2, 5, 6, 9, or 10, where the sequence file is in a FASTQ format.
16. The method of any one of embodiments 1, 5, or 9, where the first DNA polymerase is a thermostable DNA polymerase.
17. The method of embodiment 16, where the thermostable DNA polymerase is selected from the group consisting of comprising Taq DNA polymerase, Phusion® DNA polymerase, Q5C) DNA polymerase, and KAPA High Fidelity DNA polymerase.
18. The method of any one of embodiments 1, 5, or 9, where the first DNA polymerase is a non-thermostable DNA polymerase.
19. The method of embodiment 18, where the non-thermostable DNA polymerase is selected from the group consisting of phi29 DNA polymerase and Bst DNA polymerase.
20. The method of any one of embodiments 1, 5, or 9, where removing the non-extended UMI Primers in step (c) is performed by a method selected from the group consisting of solid phase reversible immobilization purification, column purification, and enzymatic digestion.
21. The method of any one of embodiments 1, 5, or 9, where removing the non-extended UMI Primers in step (c) is performed by enzymatic digestion.
22. The method of any one of embodiments 1, 2, 5, 6, 9, or 10, where a reference sequence of the at least one Target Region comprises multiple DNA sequences for each Target Region comprising single nucleotide polymorphism alleles comprising a population allele frequency of greater than 0.1%.
23. The method of any one of embodiments 1-12, where the sequence variant call further comprises removal of the NGS reads when between 1 NGS read and 100 NGS reads comprise an identical UMI sequence.
24. The method of any one of embodiments 1-12, where the sequence variant call further comprises removal of the NGS reads when the UMI sequence of the NGS reads does not comprise an appropriate degenerate base design pattern.
25. The method of any one of embodiments 1-8, where the sequence variant call further comprises:
   (a) grouping the NGS reads into at least a first UMI Family and a second UMI Family, where each NGS read within the first UMI Family comprises a first identical UMI sequence and aligns to a common amplicon, where each NGS read within the second UMI Family comprises a second identical UMI sequence and aligns to the same common amplicon, and where the UMI sequence of the first UMI Family differs by 1 nucleotide or 2 nucleotides as compared to the UMI sequence of the second UMI Family; and
   (b) removing from consideration the NGS reads in the UMI Family that has the fewest NGS reads between the first UMI Family and the second UMI Family.
26. The method of any one of embodiments 1-12, where the sequence variant call further comprises identifying a UMI Family Sequence.
27. The method of any one of embodiments 5-12, where the sequence variant call further comprises identifying one or more UMI Families comprising between 1 NGS read to 10 NGS reads comprising a sequence 100% identical to a reference sequence of the at least one Target Region.
28. The method of any one of embodiments 1-12, where the sequence variant call further comprises removal of at least one UMI Family comprising a member size smaller than X for each amplicon, where X is set as Y % of the mean value for the largest Z UMI Family size(s) in the amplicon, where Y is between 1% and 20%, and where Z is between 1 and 20.
29. The method of any one of embodiments 1-4 or 9-12, where the sequence variant call further comprises removing from consideration, for each amplicon, all NGS reads in a below-threshold UMI Family; where the below-threshold UMI Family comprises a size smaller than X, where X is Y % of the mean value for the largest Z UMI Family sizes for the amplicon, where Y is between 1% and 20%, and where Z is between 1 and 20.
30. The method of any one of embodiments 1, 5, or 9, where the set of UMI primers comprises, in order from 5′ to 3′,
   (a) a first universal region;
   (b) an optional second region comprising a length of between 1 nucleotide and 50 nucleotides;
   (c) a third region comprising a UMI sequence; and
   (d) a fourth region comprising a gene-specific sequence that is complementary to a Target Region subsequence.
31. The method of any one of embodiments 1, 5, or 9, where step (a) further comprises introduction of a set of Outer Primers, and where the second set of DNA primers introduced in step (d) comprises a set of Inner Primers, where between 3 nucleotides and 20 nucleotides positioned at the 3′ end of the Inner Primer are not subsequences of the set of Outer Primers.
32. The method of any one of embodiments 1, 5, or 9, where step (d) further comprises variant sequence enrichment.
33. The method of embodiment 32, where the variant sequence enrichment is performed by blocker displacement amplification (BDA).
34. The method of embodiment 33, where the BDA comprises amplifying a nucleic acid molecule with:
   (a) a BDA forward primer for each target genomic region, where the BDA forward primer comprises a region targeting a specific genomic region; and
   (b) a BDA blocker for each target genomic region, where 4 or more nucleotides at the 3′ end of the BDA forward primer sequence are also present at or near the 5′ end of the BDA blocker sequence, and where the BDA blocker comprises a 3′ sequence or modification that prevents extension by the DNA polymerase, and where the concentration of the BDA blocker is at least twice the concentration of the BDA forward primer.
35. The method of any one of embodiments 1, 2, 5, 6, 9, or 10, where the DNA sample comprises between 1 Target Region and 10,000 Target Regions.
36. The method of any one of embodiments 1, 2, 5, 6, 9, or 10, where the gene specific sequence is at least 90% complementary to the Target Region subsequence.
37. The method of any one of embodiments 5-8, where X, Y, and Z are the same integer for all amplicons.
38. The method of any one of embodiments 5-8, where X, Y, and Z are not the same integer for all amplicons.
39. The method of embodiments 28 or 29, where X, Y, and Z are the same integer for all amplicons.
40. The method of embodiments 28 or 29, where X, Y, and Z are not the same integer for all amplicons.

Having described the present disclosure in detail, it will be apparent that modifications, variations, and equivalent aspects are possible without departing from the spirit and scope of the present disclosure as described herein and in the appended claims. Furthermore, it should be appreciated that all examples in the present disclosure are provided as non-limiting examples.

### EXAMPLES

### Example 1. Experimental Workflow-QDBA

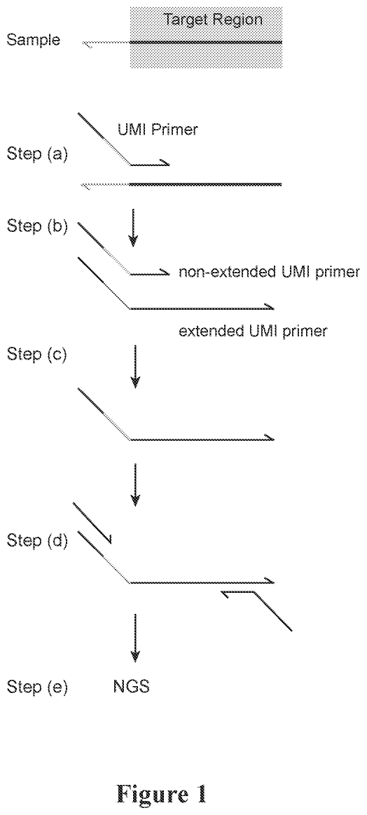

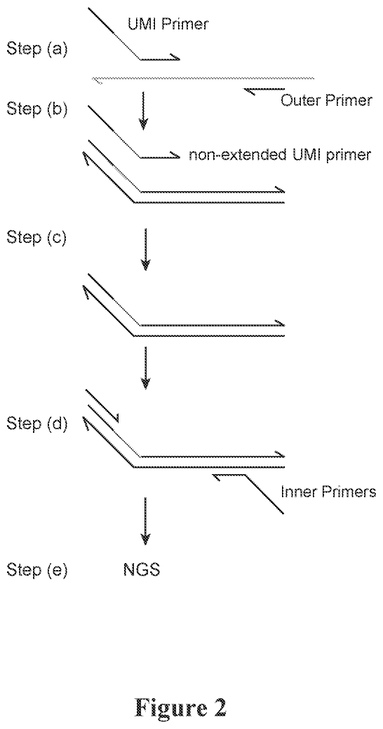

A schematic of the NGS library preparation principle is shown in FIG. 1 and FIG. 2. Two different workflows are developed based on this principle.

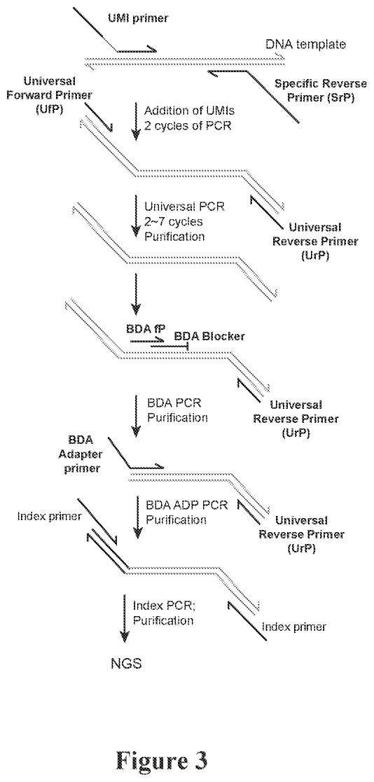

The first workflow, termed Quantitative Blocker Displacement Amplification (QBDA) as shown in FIG. 3, is combined with our previously developed BDA technology (see, for example, WO 2019/164885, which is incorporated by reference in its entirety herein) to enrich for variant sequences over wildtype (WT) sequences.

First, a unique molecular identifier (UMI) addition step is performed. A DNA sample is mixed with specific forward primers (SfP), specific reverse primers (SrP), DNA polymerase, dNTPs, and a PCR buffer.

Two cycles (not more, not less) of long-extension (about 30 minutes) PCR are performed to allow the addition of a UMI to all target loci. Each strand in one DNA molecule will carry a different UMI.

Second, a universal amplification step is performed. In order to amplify the molecules to avoid sample loss during purification while preventing addition of multiple UMIs onto the same original molecule, the annealing temperature is raised by about 8° C., and the sample is amplified for at least two cycles, and preferably about 7 cycles, using universal forward primers (UfP) and universal reverse primers (UrP). This process uses a short extension time of about 30 seconds. The addition of UfP and UrP into the reaction is performed as an open-tube step on the thermocycler. Next, purification using solid phase reversible immobilization (SPRI) magnetic beads, columns, or enzymatic digestion is carried out to remove single-stranded primers including SfP, SrP, UfP, and UrP.

Following UMI attachment, BDA amplification is performed. BDA forward primer, BDA blocker, DNA polymerase, dNTPs, and PCR buffer are mixed with the purified PCR product for BDA amplification. The BDA forward primer anneals to genomic region that is closer to SrP comparing to the region that binds to SfP. After at least two cycles, and preferably between 10 cycles and 23 cycles of BDA amplification, the PCR reaction mixture is purified by SPRI magnetic beads or columns.

Next, an adapter is added. BDA adaptor primer (comprising an Illumina adapter sequence and a BDA forward primer sequence) and UrP are mixed with the purified PCR mixture and amplified for at least 1 cycle. The adapter can also be added by enzymatic ligation reaction.

Lastly, after another purification using SPRI magnetic beads or columns, standard next generation sequencing (NGS) index PCR is performed. Libraries are normalized and loaded onto an Illumina sequencer. The NGS libraries can be sequenced by Illumina sequencer (both single-read and paired-end) or other next generation sequencers such as Ion Torrent.

All types of DNA polymerases and PCR super mixes can be used; standard annealing, extension, and denaturation temperatures for the specific DNA polymerase used for each step, except for the universal PCR step, in which the annealing temperature is raised.

Because there is variant enrichment in QBDA, low-depth sequencing is sufficient for low frequency mutation quantitation. The observed WT molecule number does not accurately reflect the real molecule number in the sample. The mutation Variant Allele Frequency (VAF) should be quantified based on the observed variant molecule number and total input molecule number. Total input molecule number is quantified by Qubit or qPCR. For example, 1 ng human genomic DNA is considered as about 290 haploid genomic equivalence (or 580 strands).

### Example 2. Experimental Workflow-QASeq

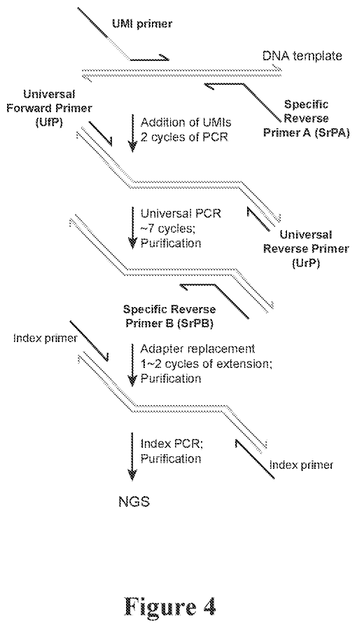

The second workflow is called Quantitative Amplicon Sequencing (QASeq), as shown in FIG. 4. There is no sequence enrichment in this method. First, a DNA sample is mixed with SfP, SrPA, DNA polymerase, dNTPs, and PCR buffer. Two cycles of long-extension (about 30 minutes) PCR are performed to allow the addition of a UMI to all target loci. Each strand in one DNA molecule will carry a different UMI.

Next, in order to amplify the molecules while preventing addition of multiple UMIs onto the same original molecule, the annealing temperature is raised by about 8° C., and the mixture is amplified for about 7 cycles using UfP and UrP. This process uses a short extension time of about 30 seconds. The addition of UfP and UrP into the reaction is performed as an open-tube step on the thermocycler.

After purification using SPRI magnetic beads or columns, SrPB primers, DNA polymerase, dNTPs, and PCR buffer are mixed with the PCR product for adapter replacement; after 2 cycles of long extension (about 30 minutes), NGS adapters are only added onto the correct PCR products, not onto primer dimers or non-specific products. Following another purification using SPRI magnetic beads or columns, standard NGS index PCR is performed. Libraries are normalized and loaded onto an Illumina sequencer.

Because there is no sequence preference in QASeq, the mutation VAF can be quantified based on the observed molecule number for variant and wildtype sequence.

### Example 3. Genotype Determination Workflow

All reads that align to the same locus are sorted by their respective UMI sequences. Reads carrying the same UMI are grouped as one UMI family. UMI family size is calculated as the number of reads comprising the same UMI, and the unique UMI number is the total count of different UMI sequences at one locus. Here, the UMI number and genotype associated with the UMIs are determined by a set of UMI correction methods: WT veto; Nearest Neighbor Check; and Dynamic Cutoff. See FIG. 5.

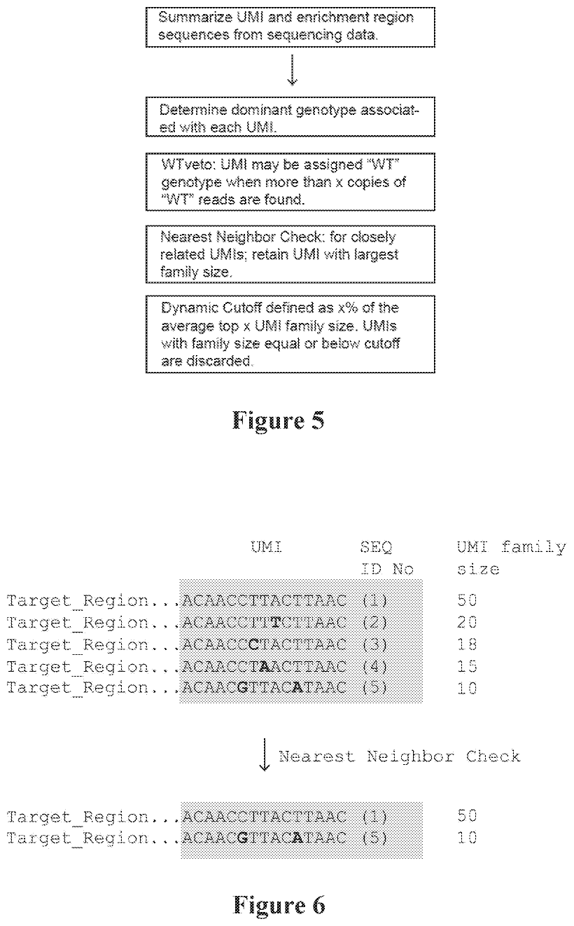

UMI families that likely resulted from PCR polymerase error or NGS sequencing error are removed from further consideration. A UMI sequence that is not consistent with a designed UMI pattern (e.g. G bases found in the poly(H) UMI sequence) are considered to be errors and are removed from further consideration. Furthermore, UMI families with high sequence similarity (Distance Threshold), such that only 1 to 2 bases are different, are deemed potential PCR artifacts. As such, a Nearest Neighbor Check is implemented to retain only the UMIs with the largest family size within groups of highly similar UMIs. See FIG. 6.

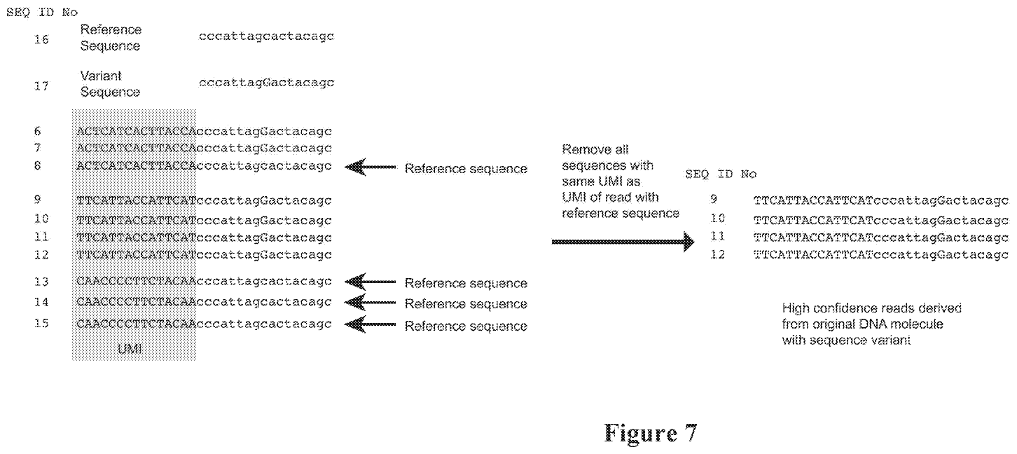

While some UMI family exhibit a single genotype, many are associated with multiple genotypes with varying frequency. We assign the dominant genotype with the most reads to each UMI family, with the following exception: where a wildtype genotype (as defined by the Human Reference Genome) is identified in x or more reads, the UMI family is assigned the wildtype genome regardless of other genotypes present. This threshold, termed WTveto, further improves the specificity of the qBDA technology (FIG. 7).

Table 1 provides a listing of the sequences found in FIG. 6 and FIG. 7.

TABLE 1
Sequences used in FIGS. 6 and 7.

| Sequence | SEQ ID NO |
|---|---|
| ACAACCTTACTTAAC | 1 |
| ACAACCTTTCTTAAC | 2 |
| ACAACCCTACTTAAC | 3 |
| ACAACCTAACTTAAC | 4 |
| ACAACGTTACATAAC | 5 |
| ACTCATCACTTACCAcccattagGactacagc | 6 |
| ACTCATCACTTACCAcccattagGactacagc | 7 |
| ACTCATCACTTACCAcccattagcactacagc | 8 |
| TTCATTACCATTCATcccattagGactacagc | 9 |
| TTCATTACCATTCATcccattagGactacagc | 10 |
| TTCATTACCATTCATcccattagGactacagc | 11 |
| TTCATTACCATTCATcccattagGactacagc | 12 |
| CAACCCCTTCTACAAcccattagcactacagc | 13 |
| CAACCCCTTCTACAAcccattagcactacagc | 14 |
| CAACCCCTTCTACAAcccattagcactacagc | 15 |
| cccattagcactacagc | 16 |
| cccattagGactacagc | 17 |

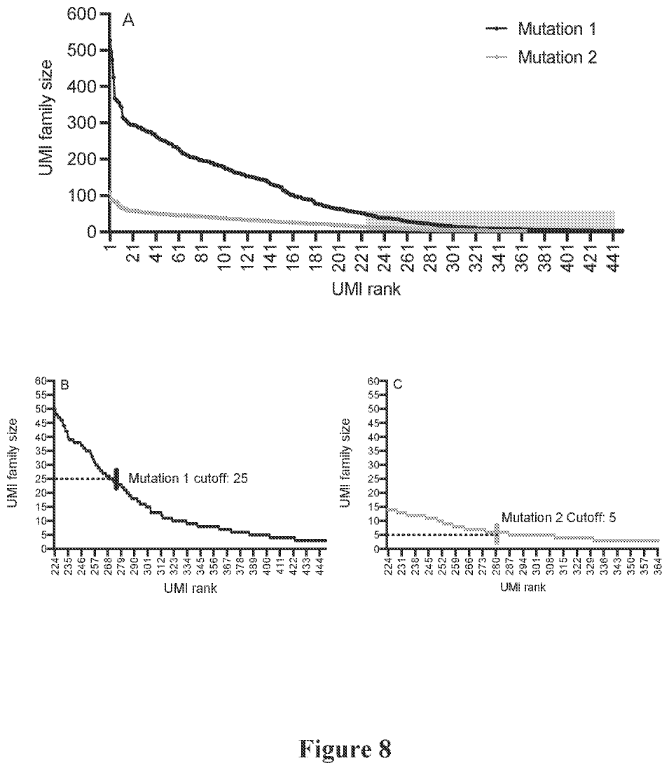

The UMI families with family sizes <Fₘᵢₙ are also removed; Fₘᵢₙ is determined based on the distribution of UMI family size. For example, Fₘᵢₙ can be set as 5% of the mean value for the largest three UMI family sizes for the target with the exact same nucleic acid sequence. See FIG. 8.

### Example 4. Non-Small Cell Lung Cancer (NSCLC) QBDA Panel

The NSCLC lung cancer panel comprises 31 BDA designs targeting hotspot mutations in 14 genes that are of clinical significance to non-small cell lung cancer. See Table 2 and Table 3.

TABLE 2
NSCLC panel enrichment regions.

| | | Enrichment Region |
|---|---|---|
| Target # | Gene | (GRCh38.p12) |
| 1 | AKT1 | Chr14: 104780200-104780218 |
| 2 | ALK | Chr2: 29222334-29222352 |
| 3 | ALK | Chr2: 29220830-29220847 |
| 4 | ALK | Chr2: 29213992-29214009 |
| 5 | ALK | Chr2: 29209816-29209832 |
| 6 | BRAF | Chr7: 140753326-140753346 |
| 7 | BRAF | Chr7: 140781595-140781614 |
| 8 | DDR2 | Chr1: 162778599-162778613 |
| 9 | EGFR | Chr7: 55174001-55174015 |
| 10 | EGFR | Chr7: 55174769-55174790 |
| 11 | EGFR | Chr7: 55181309-55181322 |
| 12 | EGFR | Chr7: 55181378-55181391 |
| 13 | EGFR | Chr7: 55191817-55191831 |
| 14 | ERBB2 | Chr17: 39724739-39724751 |
| 15 | KRAS | Chr12: 25227341-25227356 |
| 16 | KRAS | Chr12: 25245340-25245352 |
| 17 | KRAS | Chr12: 25225609-25225628 |
| 18 | MAP2K1 | Chr15: 66435106-66435124 |
| 19 | MET | Chr7: 116771829-116771852 |
| 20 | MET | Chr7: 116771974-116771998 |
| 21 | NRAS | Chr1: 114716111-114716127 |
| 22 | NRAS | Chr1: 114713907-114713924 |
| 23 | PIK3CA | Chr3: 179218294-179218311 |
| 24 | PIK3CA | Chr3: 179234281-179234301 |
| 25 | PTEN | Chr10: 87957911-87957922 |
| 26 | ROS1 | Chr6: 117318223-117318238 |
| 27 | ROS1 | Chr6: 117317175-117317190 |
| 28 | TP53 | Chr17: 7675081-7675091 |
| 29 | TP53 | Chr17: 7674879-7674896 |
| 30 | TP53 | Chr17: 7674217-7674230 |
| 31 | TP53 | Chr17: 7673802-7673816 |

TABLE 3
Oligonucleotide sequences for
the first 10 targets in the NSCLC panel.

| Identifier | Nucleic Acid Sequence | SEQ ID NO |
|---|---|---|
| SfP_NSCLC1 | GGATATTCCTTTCTACTCTTTGACATCATCTATCACGTGGCTCT | 18 |
| | CACCACCC | |
| SfP_NSCLC2 | GGATATTCCTTTCTACTCTTTGACATCATCTATCAAACAGGACG | 19 |
| | AACTGGATTTCCT | |
| SfP_NSCLC3 | GGATATTCCTTTCTACTCTTTGACATCATCTATCATCACCCCAA | 20 |
| | TGCAGCGAA | |
| SfP_NSCLC4 | GGATATTCCTTTCTACTCTTTGACATCATCTATCAGAGCACAGT | 21 |
| | CACTTTGACTCAC | |
| SfP_NSCLC5 | GGATATTCCTTTCTACTCTTTGACATCATCTATCACAGTCTTTA | 22 |
| | CTCACCTGTAGATGTCT | |
| SfP_NSCLC6 | GGATATTCCTTTCTACTCTTTGACATCATCTATCATTCATGAAG | 23 |
| | ACCTCACAGTAAAAATAGG | |
| SfP_NSCLC7 | GGATATTCCTTTCTACTCTTTGACATCATCTATCAGGAGATTCC | 24 |
| | TGATGGGCAGATTAC | |
| SfP_NSCLC8 | GGATATTCCTTTCTACTCTTTGACATCATCTATCAGTTATTCTG | 25 |
| | ATTTCCCATTCTTTTCTTTACTTA | |
| SfP_NSCLC9 | GGATATTCCTTTCTACTCTTTGACATCATCTATCATTACCTTAT | 26 |
| | ACACCGTGCCGAA | |
| SfP_NSCLC10 | GGATATTCCTTTCTACTCTTTGACATCATCTATCATCCCAGAAG | 27 |
| | GTGAGAAAGTTAAAATTC | |
| Universal Forward | CCTATGGTAGTTAAATGTACATTGGATATTCCTTTCTACTCTTT | 28 |
| Primer | GACATCATCT | |
| SrP_NSCLC1 | AGACGTGTGCTCTTCCGATCTCAATHHHHHHHHHHHHHHHCCGC | 29 |
| | TCCTTGTAGCCAATGA | |
| SrP_NSCLC2 | AGACGTGTGCTCTTCCGATCTATCAHHHHHHHHHHHHHHHCTCT | 30 |
| | CCAGGTTCTTTGGGGG | |
| SrP_NSCLC3 | AGACGTGTGCTCTTCCGATCTCAATHHHHHHHHHHHHHHHAGAT | 31 |
| | TTGCCCAGACTCAGCTC | |
| SrP_NSCLC4 | AGACGTGTGCTCTTCCGATCTATCAHHHHHHHHHHHHHHHGCTC | 32 |
| | GGGACATTGCCTGT | |
| SrP_NSCLC5 | AGACGTGTGCTCTTCCGATCTCAATHHHHHHHHHHHHHHHGCTG | 33 |
| | CCAGAAACTGCCTCT | |
| SrP_NSCLC6 | AGACGTGTGCTCTTCCGATCTCAATHHHHHHHHHHHHHHHCCAC | 34 |
| | AAAATGGATCCAGACAACTG | |
| SrP_NSCLC7 | AGACGTGTGCTCTTCCGATCTATCAHHHHHHHHHHHHHHHACAA | 35 |
| | TGTCACCACATTACATACTTACC | |
| SrP_NSCLC8 | AGACGTGTGCTCTTCCGATCTATCAHHHHHHHHHHHHHHHGACA | 36 |
| | AAAGGTGAAAGTCTCCCAC | |
| SrP_NSCLC9 | AGACGTGTGCTCTTCCGATCTATCAHHHHHHHHHHHHHHHGCTC | 37 |
| | CCAACCAAGCTCTCT | |
| SrP_NSCLC10 | AGACGTGTGCTCTTCCGATCTCAATHHHHHHHHHHHHHHHGCAA | 38 |
| | AGCAGAAACTCACATCGAG | |
| Universal Reverse | GACTGGAGTTCAGACGTGTGCTCTTCCGATCT | 39 |
| Primer | | |
| BDA_fp_NSCLC1 | CCACCCGCACGTCTGT | 40 |
| BDA_fp_NSCLC2 | GGATTTCCTCATGGAAGCCCT | 41 |
| BDA_fp_NSCLC3 | GCGAACAATGTTCTGGTGGTTG | 42 |
| BDA_fp_NSCLC4 | ACTTTGACTCACCGGTGGAT | 43 |
| BDA_fp_NSCLC5 | CGGGCCATCCCGAAGTCT | 44 |
| BDA_fp_NSCLC6 | CCTCACAGTAAAAATAGGTGATTTTGGT | 45 |
| BDA_fp_NSCLC7 | GATTACAGTGGGACAAAGAATTGGAT | 46 |
| BDA_fp_NSCLC8 | AGGGCAAGTTCACTACAGCAA | 47 |
| BDA_fp_NSCLC9 | GCCGAACGCACCGGAG | 48 |
| BDA_fp_NSCLC10 | AGAAAGTTAAAATTCCCGTCGCTAT | 49 |
| BDA_Blocker_NSCLC1 | ACGTCTGTAGGGGAGTACATCAAGACC/iSpC3//iSpC3/GA | 50 |
| BDA_Blocker_NSCLC2 | AAGCCCTGATCATCAGGTAAAGCCAC/iSpC3//iSpC3/AA | 51 |
| BDA_Blocker_NSCLC3 | TGGTGGTTGAATTTGCTGCAGAGCAGA/iSpC3//iSpC3/GA | 52 |
| BDA_Blocker_NSCLC4 | GGTGGATGAAGTGGTTTTCCTCCAA/iSpC3//iSpC3/AA | 53 |
| BDA_Blocker_NSCLC5 | CCGAAGTCTCCAATCTTGGCCACTCT/iSpC3//iSpC3/CT | 54 |
| BDA_Blocker_NSCLC6 | GTGATTTTGGTCTAGCTACAGTGAAATCTCGA/iSpC3// | 55 |
| | iSpC3/GT | |
| BDA_Blocker_NSCLC7 | AAAGAATTGGATCTGGATCATTTGGAACAGTC/iSpC3// | 56 |
| | iSpC3/cc | |
| BDA_Blocker_NSCLC8 | ACTACAGCAAGTGATGTGTGGGCCT/iSpC3//iSpC3/GA | 57 |
| BDA_Blocker_NSCLC9 | CACCGGAGCCCAGCACTTTGATC/iSpC3//iSpC3/TA | 58 |
| BDA_Blocker_ | GTCGCTATCAAGGAATTAAGAGAAGCAACA/iSpC3//iSpC3/ | 59 |
| NSCLC10 | TA | |
| Adp_fp_NSCLC1 | ACACGACGCTCTTCCGATCTCCACCCGCACGTCTGT | 60 |
| Adp_fp_NSCLC2 | ACACGACGCTCTTCCGATCTGGATTTCCTCATGGAAGCCCT | 61 |
| Adp_fp_NSCLC3 | ACACGACGCTCTTCCGATCTGCGAACAATGTTCTGGTGGTTG | 62 |
| Adp_fp_NSCLC4 | ACACGACGCTCTTCCGATCTACTTTGACTCACCGGTGGAT | 63 |
| Adp_fp_NSCLC5 | ACACGACGCTCTTCCGATCTCGGGCCATCCCGAAGTCT | 64 |
| Adp_fp_NSCLC6 | ACACGACGCTCTTCCGATCTCCTCACAGTAAAAATAGGTGATTT | 65 |
| | TGGT | |
| Adp_fp_NSCLC7 | ACACGACGCTCTTCCGATCTGATTACAGTGGGACAAAGAATTGG | 66 |
| | AT | |
| Adp_fp_NSCLC8 | ACACGACGCTCTTCCGATCTAGGGCAAGTTCACTACAGCAA | 67 |
| Adp_fp_NSCLC9 | ACACGACGCTCTTCCGATCTGCCGAACGCACCGGAG | 68 |
| Adp_fp_NSCLC10 | ACACGACGCTCTTCCGATCTAGAAAGTTAAAATTCCCGTCGCTA | 69 |
| | T | |

The positive control consists of synthetic double-stranded gBlocks harboring clinical mutations corresponding to each enrichment region present at 0.35-2.8% VAF in a wildtype genomic DNA background. See Table 4. The NSCLC QBDA panel detected mutations in the positive control within 2-fold of expected VAF in 90% of all BDA amplicons. See Table 4.

TABLE 4
NSCLC panel gBlock spike-in standards quantitation results.

| | | gBlock spike-in | | Spike-in | Observed |
|---|---|---|---|---|---|
| Target # | Gene | AA mutation | Cosmic ID | VAF (%) | VAF (%) |
| 1 | AKT1 | E17K | COSV62571334 | 2.80 | 3.03 |
| 2 | ALK | I1171N | COSV66556242 | 0.74 | 0.41 |
| 3 | | F1174V | COSV66559314 | 0.66 | 0.69 |
| 4 | | F1245C | COSV66563458 | 0.76 | 0.74 |
| 5 | | G1269A | COSV66557991 | 1.14 | 1.64 |
| 6 | BRAF | V600D | COSV56059623 | 1.00 | 0.80 |
| 7 | | G469V | COSV56062352 | 0.77 | 1.10 |
| 8 | DDR2 | S768R | COSV63371628 | 0.77 | 0.78 |
| 9 | EGFR | G719A | COSV51769339 | 1.06 | 0.78 |
| 10 | | A750P | COSV51830861 | 0.56 | 0.36 |
| 11 | | S768I | COSV51768106 | 0.62 | 0.79 |
| 12 | | T790M | COSV51765492 | 0.67 | 0.72 |
| 13 | | L861Q | COSV51766344 | 0.83 | 0.85 |
| 14 | ERBB2 | G776delinsLC | COSV54077923 | 1.75 | 1.06 |
| 15 | KRAS | Q61H | COSV55498802 | 0.83 | 2.50 |
| 16 | | G12D | COSV55497369 | 1.25 | 0.90 |
| 17 | | A146P | COSV55541748 | 0.76 | 1.04 |
| 18 | MAP2K1 | Q56P | COSV61068787 | 0.71 | 1.26 |
| 19 | MET | exon 14 skipping | — | 0.35 | 1.04 |
| 20 | | exon 14 skipping | — | 0.80 | 0.15 |
| 21 | NRAS | G12S | COSV54736621 | 1.03 | 0.81 |
| 22 | | Q61K | COSV54736310 | 0.94 | 1.11 |
| 23 | PIK3CA | E542K | COSV55873227 | 0.75 | 0.60 |
| 24 | | H1047L | COSV55873401 | 0.74 | 1.06 |
| 25 | PTEN | R233* | COSV64288653 | 1.18 | 0.71 |
| 26 | ROS1 | S1986F | COSV63862079 | 1.34 | 1.02 |
| 27 | | G2032R | COSV63851612 | 0.93 | 0.89 |
| 28 | TP53 | R175H | COSV52661038 | 0.62 | 0.55 |
| 29 | | R213* | COSV52665560 | 0.79 | 0.70 |
| 30 | | G245S | COSV52661877 | 1.01 | 1.00 |
| 31 | | R273H | COSV52660980 | 1.10 | 0.67 |

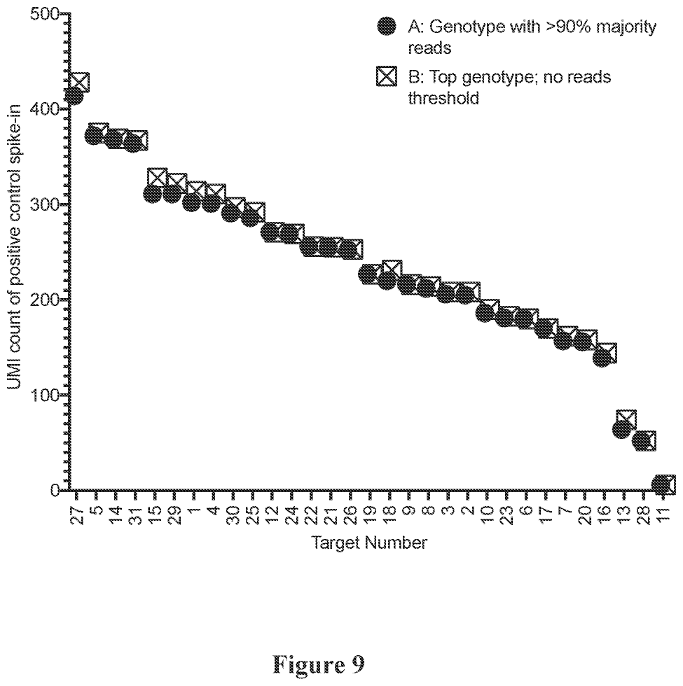

Using the NSCLC QBDA design as prototype, two methods of UMI genotype assignment are compared. Simply assigning the dominant genotype to each UMI resulted in UMI counts of the positive control spike-in comparable to requiring reads associated with the dominant genotype to exceed a fixed threshold, e.g. 90%, of total reads. See FIG. 9.

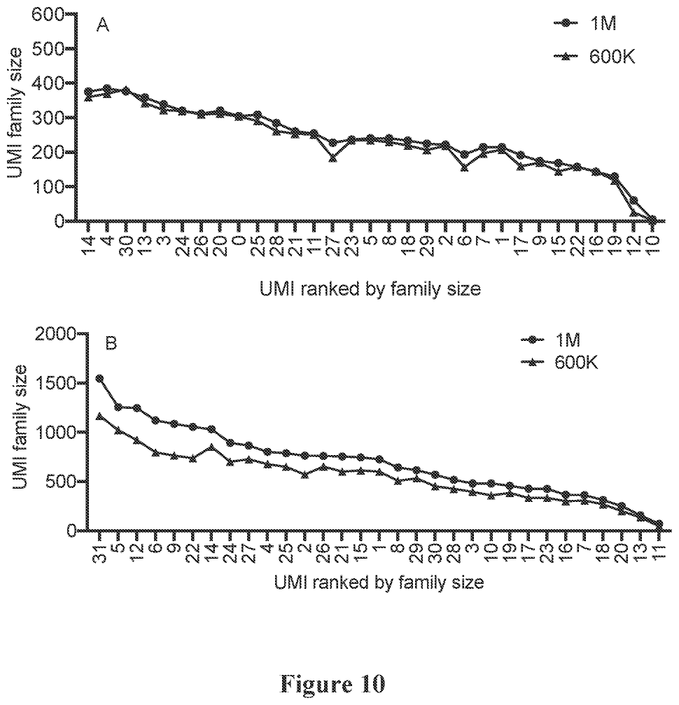

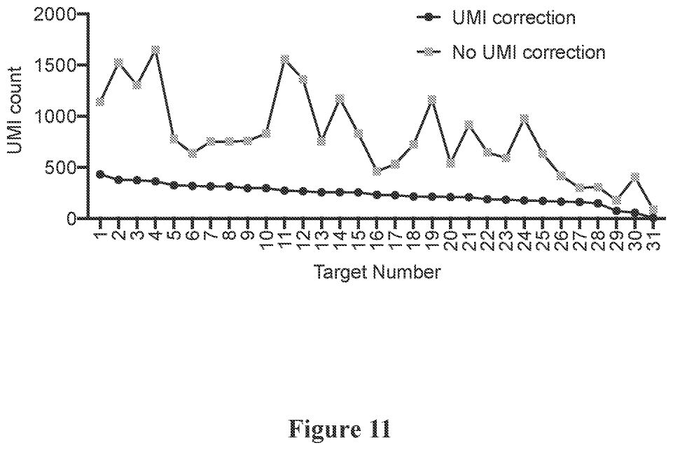

Furthermore, Dynamic Cutoff eliminated the effect of sequencing read depth on UMI count quantification. See FIG. 10. Together, the application of UMI correction improved UMI quantification by avoiding over-estimation due to variable effects PCR error, sequencing error, and sequencing depth bias. See FIG. 11.

### Example 5. Alternative QBDA Experimental Workflow

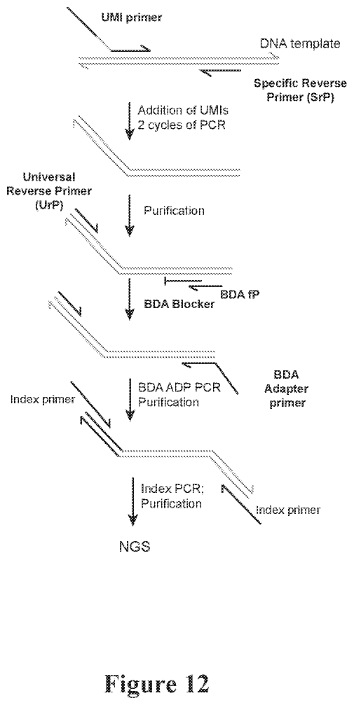

The alternative QBDA workflow (FIG. 12) consists of only four subsequent PCR reactions. The first reaction labels each target molecule with UMI sequences and is followed by a magnetic bead purification (SPRI) step to remove unreacted primers and byproducts. This first purification is carried out by adding 200 ng of carrier RNA acting as passivating agent solution before subjecting the sample to SPRI. Next, a second reaction (BDA-PCR) is carried out, without purification, and it is immediately followed by a third PCR reaction that attaches sequencing primers (adapters). After a second SPRI purification, a fourth reaction attaches Illumina's grafting sequences and indexes. Finally, an SPRI purification step purifies the library before NGS.

Comparing to the standard QBDA protocol shown in FIG. 3, the simplified workflow eliminates the universal PCR amplification step and eliminates the purification step after BDA amplification.

The quantitation performance from alternative QBDA workflow is similar to standard QBDA in a positive control sample that contains variants for each amplicon at ˜1% VAF. See Table 5.

TABLE 5
Experimental results comparison between
standard and simplified QBDA workflow.

| | | | | Experimental | Experimental |
|---|---|---|---|---|---|
| | | | | VAF % from | VAF % from |
| | | Variant | | alternative | standard |
| Target # | Gene | spike-in | Variant found | protocol | QBDA |
| 1 | MAP2K1 | 2A > G | 2A > G | 1.5 | 0.9 |
| 2 | | 2T > A | 2T > A | 1.4 | 0.9 |
| 3 | | 12C > T | 12C > T | 1.1 | 1.1 |
| 4 | MAP2K2 | 14A > C | 14A > C | 0.3 | 1.2 |
| 5 | | 3T > A | 3T > A | 1.1 | 1.2 |
| 6 | | 2G > A | 2G > A | 0.8 | 0.8 |
| 7 | AKT1 | 4C > A | 4C > A | 1.3 | 1.2 |
| 8 | AKT3 | 16C > T | 16C > T | 2.1 | 0.5 |
| 9 | NRAS | 5C > T | 5C > T | 0.9 | 0.6 |
| 10 | | 4C > A | 4C > A | 1.4 | 1.0 |
| 11 | KRAS | 6C > T | 6C > T | 2.2 | 1.7 |
| 12 | | 3TC > AA | 3TC > AA | 0.9 | 0.7 |
| 13 | PIK3CA | 4G > A | 4G > A | 2.7 | 2.0 |
| 14 | | 4C > T | 4C > T | 0.4 | 0.8 |
| 15 | BRAF | 4A > T | 4A > T | 0.8 | 1.0 |

1. A method for analyzing a DNA sample comprising at least one Target Region for potential sequence variants, the method comprising:
(a) contacting the DNA sample with:
(i) a set of unique molecular identifier (UMI) Primers, wherein each UMI Primer comprises a UMI sequence and a gene-specific sequence that is complementary to a Target Region subsequence;
(ii) a first DNA polymerase; and
(iii) reagents and buffers needed for DNA polymerase extension to generate a mixture;
(b) subjecting the mixture of step (a) to one or more temperatures that allow primer binding and DNA polymerase extension;
(c) removing non-extended UMI Primers to produce a product;
(d) mixing the product of step (c) with:
(i) a second set of DNA primers;
(ii) a second DNA polymerase; and
(iii) reagents and buffers needed for a polymerase chain reaction (PCR),
and performing PCR to produce a PCR product;
(e) subjecting the PCR product produced in step (d) to high-throughput DNA sequencing and obtaining a sequence file comprising next generation sequencing (NGS) reads;
(f) identifying a vetoed UMI sequence, wherein at least 1, 2, 3, 4, 5, 6, 7, 8, 9, or NGS reads containing the vetoed UMI sequence also comprise a wildtype sequence of the at least one Target Region;
(g) removing from consideration all NGS reads comprising the vetoed UMI sequence identified in step (0; and
(h) generating a sequence variant call by quantifying DNA variant molecules based on bioinformatic analysis of the NGS reads that are not removed in step (g).

2. A method for analyzing a DNA sample comprising at least one Target Region for potential sequence variants, the method comprising:
(a) contacting the DNA sample with:
(i) a set of unique molecular identifier (UMI) Primers, wherein each UMI Primer comprises a UMI sequence and a gene-specific sequence that is complementary to a Target Region subsequence;
(ii) a first DNA polymerase; and
(iii) reagents and buffers needed for DNA polymerase extension to generate a mixture;
(b) subjecting the mixture of step (a) to temperatures that allow primer binding and DNA polymerase extension;
(c) removing non-extended UMI Primers to produce a product;
(d) mixing the product of (c) with:
(i) a second set of DNA primers;
(ii) a second DNA polymerase; and
(iii) reagents and buffers needed for a polymerase chain reaction (PCR),
and performing PCR to produce a PCR product;
(e) subjecting the PCR product produced in step (d) to high-throughput DNA sequencing and obtaining a sequence file comprising next generation sequencing (NGS) reads;
(f) grouping the NGS reads into at least one UMI Family, wherein each NGS read within a UMI Family comprises an identical UMI sequence and aligns to the same amplicon;
(g) removing from consideration, for each amplicon, all NGS reads in a below-threshold UMI Family; wherein the below-threshold UMI Family comprises a size smaller than X, wherein X is Y % of the mean value for the largest Z UMI Family sizes for the amplicon, wherein Y is between 1% and 20%, and wherein Z is between 1 and 20; and
(h) generating a sequence variant call based on bioinformatic analysis of the NGS reads that were not removed in step (g).

3. A method for analyzing a DNA sample comprising at least one Target Region for potential sequence variants, the method comprising:
(a) contacting the DNA sample with:
(i) a set of unique molecular identifier (UMI) Primers, wherein each UMI Primer comprises a UMI sequence and a gene-specific sequence that is complementary to a Target Region subsequence;
(ii) a first DNA polymerase; and
(iii) reagents and buffers needed for DNA polymerase extension to generate a mixture;
(b) subjecting the mixture of step (a) to temperatures that allow primer binding and DNA polymerase extension;
(c) removing non-extended UMI Primers to produce a product;
(d) mixing the product of (c) with:
(i) a second set of DNA primers;
(ii) a second DNA polymerase; and
(iii) reagents and buffers needed for a polymerase chain reaction (PCR),
and performing PCR to produce a PCR product;
(e) subjecting the PCR product produced in step (d) to high-throughput DNA sequencing and obtaining a sequence file comprising next generation sequencing (NGS) reads;
(f) grouping the NGS reads into at least a first UMI Family and a second UMI Family, wherein each NGS read within the first UMI Family comprises a first identical UMI sequence and aligns to a common amplicon, wherein each NGS read within the second UMI Family comprises a second identical UMI sequence and aligns to the common amplicon, and wherein the UMI sequence of the first UMI Family differs by 1 nucleotide or 2 nucleotides as compared to the UMI sequence of the second UMI Family;
(g) removing from consideration the NGS reads in the UMI Family that has the fewest NGS reads between the first UMI Family and the second UMI Family; and
(h) generating a sequence variant call based on bioinformatic analysis of the NGS reads that were not removed in step (g).

4. The method of any one of claims 1-3, wherein the UMI sequence comprises between 7 degenerate nucleotides and 30 degenerate nucleotides, and wherein each degenerate nucleotide is selected from the group consisting of N, B, D, H, V, S, W, Y, R, M, and K.

5. The method of any one of claims 1-3, wherein the high-throughput DNA sequencing comprises sequencing-by-synthesis or nanopore-based sequencing.

6. The method of any one of claims 1-3, wherein the sequence file is in a FASTQ format.

7. The method of any one of claims 1-3, wherein the first DNA polymerase is a thermostable DNA polymerase.

8. The method of claim 7, wherein the thermostable DNA polymerase is selected from the group consisting of comprising Taq DNA polymerase, Phusion® DNA polymerase, Q5C) DNA polymerase, and KAPA High Fidelity DNA polymerase.

9. The method of any one of claims 1-3, wherein the first DNA polymerase is a non-thermostable DNA polymerase.

10. The method of claim 9, wherein the non-thermostable DNA polymerase is selected from the group consisting of phi29 DNA polymerase and Bst DNA polymerase.

11. The method of any one of claims 1-3, wherein removing the non-extended UMI Primers in step (c) is performed by a method selected from the group consisting of solid phase reversible immobilization purification, column purification, and enzymatic digestion.

12. The method of any one of claims 1-3, wherein removing the non-extended UMI Primers in step (c) is performed by enzymatic digestion.

13. The method of any one of claims 1-3, wherein a reference sequence of the at least one Target Region comprises multiple DNA sequences for each Target Region comprising single nucleotide polymorphism alleles comprising a population allele frequency of greater than 0.1%.

14. The method of any one of claims 1-3, wherein the sequence variant call further comprises removal of the NGS reads when between 1 NGS read and 100 NGS reads comprise an identical UMI sequence.

15. The method of any one of claims 1-3, wherein the sequence variant call further comprises removal of the NGS reads when the UMI sequence of the NGS reads does not comprise an appropriate degenerate base design pattern.

16. The method of claim 1 or 2, wherein the sequence variant call further comprises:
(a) grouping the NGS reads into at least a first UMI Family and a second UMI Family, wherein each NGS read within the first UMI Family comprises a first identical UMI sequence and aligns to a common amplicon, wherein each NGS read within the second UMI Family comprises a second identical UMI sequence and aligns to the same common amplicon, and wherein the UMI sequence of the first UMI Family differs by 1 nucleotide or 2 nucleotides as compared to the UMI sequence of the second UMI Family; and
(b) removing from consideration the NGS reads in the UMI Family that has the fewest NGS reads between the first UMI Family and the second UMI Family.

17. The method of any one of claims 1-3, wherein the sequence variant call further comprises identifying a UMI Family Sequence.

18. The method of claim 2 or 3, wherein the sequence variant call further comprises identifying one or more UMI Families comprising between 1 NGS read to 10 NGS reads comprising a sequence 100% identical to a reference sequence of the at least one Target Region.

19. The method of any one of claims 1-3, wherein the sequence variant call further comprises removal of at least one UMI Family comprising a member size smaller than X for each amplicon, wherein X is set as Y % of the mean value for the largest Z UMI Family size(s) in the amplicon, wherein Y is between 1% and 20%, and wherein Z is between 1 and 20.

20. The method of claim 1 or 3, wherein the sequence variant call further comprises removing from consideration, for each amplicon, all NGS reads in a below-threshold UMI Family; wherein the below-threshold UMI Family comprises a size smaller than X, wherein X is Y % of the mean value for the largest Z UMI Family sizes for the amplicon, wherein Y is between 1% and 20%, and wherein Z is between 1 and 20.

21. The method of any one of claims 1-3, wherein the set of UMI primers comprises, in order from 5′ to 3′,
(a) a first universal region;
(b) an optional second region comprising a length of between 1 nucleotide and 50 nucleotides;
(c) a third region comprising a UMI sequence; and
(d) a fourth region comprising a gene-specific sequence that is complementary to a Target Region subsequence.

22. The method of any one of claims 1-3, wherein step (a) further comprises introduction of a set of Outer Primers, and wherein the second set of DNA primers introduced in step (d) comprises a set of Inner Primers, wherein between 3 nucleotides and 20 nucleotides positioned at the 3′ end of the Inner Primer are not subsequences of the set of Outer Primers.

23. The method of any one of claims 1-3, wherein step (d) further comprises variant sequence enrichment.

24. The method of claim 23, wherein the variant sequence enrichment is performed by blocker displacement amplification (BDA).

25. The method of claim 24, wherein the BDA comprises amplifying a nucleic acid molecule with:
(a) a BDA forward primer for each target genomic region, wherein the BDA forward primer comprises a region targeting a specific genomic region; and
(b) a BDA blocker for each target genomic region, wherein 4 or more nucleotides at the 3′ end of the BDA forward primer sequence are also present at or near the end of the BDA blocker sequence, and wherein the BDA blocker comprises a 3′ sequence or modification that prevents extension by the DNA polymerase, and wherein the concentration of the BDA blocker is at least twice the concentration of the BDA forward primer.

26. The method of any one of claims 1-3, wherein the DNA sample comprises between 1 Target Region and 10,000 Target Regions.

27. The method of any one of claims 1-3, wherein the gene specific sequence is at least 90% complementary to the Target Region subsequence.

28. The method of claim 2, wherein X, Y, and Z are the same integer for all amplicons.

29. The method of claim 2, wherein X, Y, and Z are not the same integer for all amplicons.

30. The method of claim 19 or 20, wherein X, Y, and Z are the same integer for all amplicons.

31. The method of claim 19 or 20, wherein X, Y, and Z are not the same integer for all amplicons.
